SEQUENCE LISTING (61 sequences)

SEQ ID NO: 1 - DNA, 1553 bp, Artificial Sequence
  Aac-aphD resistance marker + P-drfa
acagaagactcctttttgttaaaattatactataaattcaactttgcaacagaacctcttttctagtataatgtttattaatatagtacatatttttataaggagttggatccatgaatatagttgaaaatgaaatatgtataagaactttaatagatgatgattttcctttgatgttaaaatggttaactgatgaaagagtattagaattttatggtggtagagataaaaaatatacattagaatcattaaaaaaacattatacagagccttgggaagatgaagtttttagagtaattattgaatataacaatgttcctattggatatggacaaatatataaaatgtatgatgagttatatactgattatcattatccaaaaactgatgagatagtctatggtatggatcaatttataggagagccaaattattggagtaaaggaattggtacaagatatattaaattgatttttgaatttttgaaaaaagaaagaaatgctaatgcagttattttagaccctcataaaaataatccaagagcaataagggcataccaaaaatctggttttagaattattgaagatttgccagaacatgaattacacgagggcaaaaaagaagattgttatttaatggaatatagatatgatgataatgccacaaatgttaaggcaatgaaatatttaattgagcattactttgataatttcaaagtagatagtattgaaataatcggtagtggttatgatagtgtggcatatttagttaataatgaatacatttttaaaacaaaatttagtactaataagaaaaaaggttatgcaaaagaaaaagcaatatataattttttaaatacaaatttagaaactaatgtaaaaattcctaatattgaatattcgtatattagtgatgaattatctatactaggttataaagaaattaaaggaacttttttaacaccagaaatttattctactatgtcagaagaagaacaaaatttgttaaaacgagatattgccagttttttaagacaaatgcacggtttagattatacagatattagtgaatgtactattgataataaacaaaatgtattagaagagtatatattgttgcgtgaaactatttataatgatttaactgatatagaaaaagattatatagaaagttttatggaaagactaaatgcaacaacagtttttgagggtaaaaagtgtttatgccataatgattttagttgtaatcatctattgttagatggcaataatagattaactggaataattgattttggagattctggaattatagatgaatattgtgattttatatacttacttgaagatagtgaagaagaaataggaacaaattttggagaagatatattaagaatgtatggaaatatagatattgagaaagcaaaagaatatcaagatatagttgaagaatattatcctattgaaactattgtttatggaattaaaaatattaaacaggaatttatcgaaaatggtagaaaagaaatttataaaaggacttataaagattga

SEQ ID NO: 2 - DNA, 2475 bp, Artificial Sequence
  Spo0A knock-out cassette
gtttccggctactaaaggaatggttattaaaattacagatcccaaattgctaaagaaaacaggtggaattgttcaaggaatgagcgggagcccgattattcaaaacggtaaagtcgttggagcggttacccatgtttttgtaaatgatcctacctcgggttacggtgtccatattgaatggatgcttaacgaagcaggaatcgatatttataaaaataatcgaaaaaaagcgagctaatgttgtcattagttcgctttttttgataaaaattattgtcgtaaatgtaagagaaaaacgtcgaaaaacaaagaaaataagagaaaatcgaataaattccatttaaaagacttttttctaaaaaaataaaggaaataatgttgcattgtcgaatttgtcctttagaataaagaatagagacacgtaggaaacgacgtataacataaagggcatgctcaatctttataagtccttttataaatttcttttctaccattttcgataaattcctgtttaatatttttaattccataaacaatagtttcaataggataatattcttcaactatatcttgatattcttttgctttctcaatatctatatttccatacattcttaatatatcttctccaaaatttgttcctatttcttcttcactatcttcaagtaagtatataaaatcacaatattcatctataattccagaatctccaaaatcaattattccagttaatctattattgccatctaacaatagatgattacaactaaaatcattatggcataaacactttttaccctcaaaaactgttgttgcatttagtctttccataaaactttctatataatctttttctatatcagttaaatcattataaatagtttcacgcaacaatatatactcttctaatacattttgtttattatcaatagtacattcactaatatctgtataatctaaaccgtgcatttgtcttaaaaaactggcaatatctcgttttaacaaattttgttcttcttctgacatagtagaataaatttctggtgttaaaaaagttcctttaatttctttataacctagtatagataattcatcactaacatacgaatattcaatattaggaatttttacattagtttctaaatttgtatttaaaaaattatatattgctttttcttttgcataaccttttttcttattagtactaaattttgttttaaaaatgtattcattattaactaaatatgccacactatcataaccactaccgattatttcaatactatctactttgaaattatcaaagtaatgctcaattaaatatttcattgccttaacatttgtggcattatcatcatatctatattccattaaataacaatcttcttttttgccctcgtgtaattcatgttctggcaaatcttcaataattctaaaaccagatttttggtatgcccttattgctcttggattatttttatgagggtctaaaataactgcattagcatttctttcttttttcaaaaattcaaaaatcaatttaatatatcttgtaccaattcctttactccaataatttggctctcctataaattgatccataccatagactatctcatcagtttttggataatgataatcagtatataactcatcatacattttatatatttgtccatatccaataggaacattgttatattcaataattactctaaaaacttcatcttcccaaggctctgtataatgtttttttaatgattctaatgtatattttttatctctaccaccataaaattctaatactctttcatcagttaaccattttaacatcaaaggaaaatcatcatctattaaagttcttatacatatttcattttcaactatattcatggatccaactccttataaaaatatgtactatattaataaacattatactagaaaagaggttctgttgcaaagttgaatttatagtataattttaacaaaaaggagtcttctgtggtaccgcatgcggggttttgagtgagctgataccttttgtaatacagagtacatgccgataaatttttggaatttacactagatagtaaactgaaaaccaataaactttttactgctgggccgatgaattttactcattcattggtctttttattttgttttaagaggtgaaaatattgaggttaatggaacgtacatcttttttttcttcatatccataatggagtattttattgaaataatttatttaattgttatgagggttttgtgtttgaaagaagatttagaagatgcattgaaaaattcatctccatattttaaatgggatatagataagtaaattataaaaaagcagccttcagaaaaggctgctttttttaatccattaatttagcaatcggttttgaaaggaacagaacaataattccaagtacaattgttacagctccgattaagctgaagatatcg

SEQ ID NO: 3 - DNA, 848 bp, Artificial Sequence
  ermC resistance marker + P-drfa promoter
acagaagactcctttttgttaaaattatactataaattcaactttgcaacagaacctcttttctagtataatgtttattaatatagtacatatttttataaggagttggatccatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctagagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaa

SEQ ID NO: 4 - DNA, 2995 bp, Artificial Sequence
  xylAR knockout cassette
gagaactgcttctctcattactttcgtctctgcaagaatcacttattctacaaagagaaaaagtagccattgaatatagaggggataatcagaagatatttcaagaacagatgatctttatgctgaactttgcagagcaaaacaaaattcctgaaatgattgaaagtacactcgttttaaatgaagttaaaacatttcattatatagaggaattaaaagctgcaacattcaactggatatatgaacgttttcagcagatttttccgcctaagacttctccttatttatttgatggagctgtgctgctagaaggaatgcttaacaatatgtttaaaatgaacaaagcgctgaactatccattacctattgaacaaattgtccgttattgcatgaagcgtacagaggaaatcataatcgaagtagcagaacaaaaagaacagttatttaacccttccgtttactctgcggtatttaatgagaaaacagaaagtcatttttcgacagatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaaggtaccacagaagactcctttttgttaaaattatactataaattcaactttgcaacagaacctcttttctagtataatgtttattaatatagtacatatttttataaggagttggatccatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctagagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaacccgggaagacaaacgagagtagaaacaatttctactctcgtttataaacgcggaggtatcaagatgaaatatgtaattggaattgatctaggaacaagcgccgtaaaaacggttttagttgatacaaaaggcaagatttgcgctgtggtttctaagaattatcctctccttcatgaaaaaacagggtatagcgaacagaatccagaagaatgggttcagcagacggctagtgctttaagtgaattaattgctaacgctaatataaaacccgaagatatagaaggaattagttattctggacaaatgcatggccttgtgatgctggatgaaaactacaacctattacgtaacgccattttgtggaatgacacaagaacaacagaacaatgtcatcatctctataaagttattggagaaaaacgtcttttaaagataacaaaaaaccaggctttagaaggatttactcttcctaagttattgtgggtaaaagagtatgaaccggctatatttgaaaaaacagctgtatttatgctgcctaaagattatttacgctttaaaatgaccggctttattcatagtgaatattcagatgctgccggtacattattattaaacatcagcaagaaaaaatgggatgatgagctttgccaattagcaggaatcaatattaatctttgtccgccactagttgaatctagtgaatgcataggcaatattagtgaagcgttctctcaaaaaacgggattagccacttcaacgaaagtatttgcaggtggagctgataatgcttgcggagcaattggcgccggtattttatcatcaggggaaacactatgtagtattgggacatcgggtgttgttctttcttatgaggaacgaagcgatagagactttgatggaaaagtccattattttaatcatggaaaagaaaatgccttttatacgatgggtgttacgctatctgcggggcacagtttaggctggtttaaggacaatttcgcaaaaaacgtcacatttgaagaattattggctgacctaaaaaatgtcccaatcggctcaaacggccttttatttaccccttacttagtcggggaacgtacaccgcatgcagatgctacg

SEQ ID NO: 5 - DNA, 2041 bp, Artificial Sequence
  leuCD knockout cassette
gctacagctgcatctgactcttctccttgcatcatcagactaacagcatacaccgctgctgtccaaagttttacattatcacccggtaaaaacggaaatgtaataaataaatgacgctgccatatttcaacggcaacctgcagcaatgtaggattttcatgctccagcttttccgtcagctgtgcttctacttgctcggcaaacaaatacggtgttgttcgttgaagctgaagcgcatttacttttttcgtctctccaaacttagtaatcataaaatcaatgtctacttcttcttccataactgtttttaaagcgactgtttttacgatagggtgaacagcttcagatgaaaatagctgttccagaaagacgcgctgcttttcaattctctttgtttttaatgaatgaacccattcaatctgatcctgaatattgtctacagctgtgaatgatttaaactgctctagctgttcttcttctgtttgttcttctatgacttgttcttgattcattctattcttgcagaactccaatagctgataaagctgttcagcatgttcagcagggagtttgtgttcttgaataactgcttgtagagtgacatatccttcatgatagtgaccaagctgaattaacaccatgaggtacatttgcaaattatgataataatcgccaatatcttctttcagcatttgctcacataattctttagctatgcctgcatcaccgagctccacgtgacaaataacaaggccaagcaagatttcatgatgcttattatcaagttcgtaggcctgctcaaataaaggaagtgcttcttgaaattgcttgtgctgcatatgatctgtagctttttcaaccaaccgttcttttaatagaggaaaagctacaattttgttttgttgttcattcgacatgttttctccccacttcgcggtccatttgctttacagtttagcaattcggttaacggaacgcaaaaaaatcgagcaaaattataattttgctcgatttttggggatccactgctcctcctttctgattatgaaaaacgtttctcacaaaacgtgagaaacgcatgattaactgtaagctgtcataatgcttgaaatggcagaatcattggcaattaccgcagttatcgcctccaccattttatcggtacctaaataatgattaccgtctgctaagtcagctgttcgatgacctgcatgtaatacactttcaaccgcttgttcaatcactgctgcttctttttctaatccaaacgaatgtctaagcaacattgctgcagataaaatagcagcaatcggattagctttgttttgaccagcaatatctggtgcagaaccgtgaaccggttcgtatagagacggaccatcagccgttaagctcgcagatggcaacattcctaacgatcctgtcagcatagaagcctcatcactcaaaatatcaccaaacatattttcagttaccactacatcaaaatacttcggattacgaattaactgcatagcagctgaatctacaagcatatgctcaagctctacatccgggtagtcttttgctacttcttcagctacttcacgccacactctactagactcaagcacattcgctttatcaacagacgttacttttttacgtcgatttacagccgtgtcaaatgcttggcgaataattctttctatttctgtacgcttataaaataatgtgtcgacaactgtttctttatttccttctgttcggcgctcgctaggcttaccaaaatataagcctcctgttagctcacggacgataatgaagtcaacaccttcaatgtattcttttttcagaggagaggcatccgctaagctatcataaaacgtcactggacgaagattagcatataaatccagttcttttcgaatagctagcaaacctttttccggtcgcaaatgtgaagggttacgatcccattttggccctccaaccgatccaagtaatacagcatccgcctgcttacaagtctcaattgtactttctggaagaggtgtgcccg

SEQ ID NO: 6 - DNA, 469 bp, Bacillus megaterium
ccgcggagtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgaaattcagtaagggtgaccgagtgtacttccaaagcactcgttatcaaaaagaatgtacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgta

SEQ ID NO: 7 - DNA, 316 bp, Artificial Sequence
  Pleu503
ccgcggagtggttggctatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaaaaagacttttcgccccttgacctatatcatttgatagggtctatttaggggtgagaagtcttttttcttttatctagaaaacgaaaggatgagaaaatacgta

SEQ ID NO: 8 - DNA, 236 bp, Artificial Sequence
  Pleu201
ccgcggagtggttgactatgcatagaaatgggcgtaagatagtcctcaaatataagttttctaaaaatttacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgta

SEQ ID NO: 9 - DNA, 439 bp, Artificial Sequence
  Pleu306
ccgcggagtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgcaaagcactcgttatcaaaaagaatgtacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgta

SEQ ID NO: 10 - DNA, 2086 bp, Artificial Sequence
  leuCD + transcriptional terminator
atgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccgaattgctaaa

SEQ ID NO: 11 - DNA, 2306 bp, Artificial Sequence
  P201 + leuCD + transcriptional terminator
agtggttgactatgcatagaaatgggcgtaagatagtcctcaaatataagttttctaaaaatttacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccg

SEQ ID NO: 12 - DNA, 2539 bp, Artificial Sequence
  Pleu + leuCD + transcriptional terminator
agtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgaaattcagtaagggtgaccgagtgtacttccaaagcactcgttatcaaaaagaatgtacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccg

SEQ ID NO: 13 - DNA, 2386 bp, Artificial Sequence
  P503 + leuCD + transcriptional terminator
agtggttggctatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccg

SEQ ID NO: 14 - DNA, 2509 bp, Artificial Sequence
  P306 + leuCD + transcriptional terminator
agtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgcaaagcactcgttatcaaaaagaatgtacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccg

SEQ ID NO: 15 - DNA, 38 bp, Artificial Sequence
  PCR primer
aaccggtttgtcgacgtttccggctactaaaggaatgg

SEQ ID NO: 16 - DNA, 38 bp, Artificial Sequence
  PCR primer
aaccggtttgcatgccctttatgttatacgtcgtttcc

SEQ ID NO: 17 - DNA, 38 bp, Artificial Sequence
  PCR primer
aaccggtttgcatgcggggttttgagtgagctgatacc

SEQ ID NO: 18 - DNA, 38 bp, Artificial Sequence
  PCR primer
aaccggtttgaattccgatatcttcagcttaatcggag

SEQ ID NO: 19 - DNA, 39 bp, Artificial Sequence
  PCR primer
aaccggtttgcatgcggtaccacagaagactcctttttg

SEQ ID NO: 20 - DNA, 45 bp, Artificial Sequence
  PCR primer
aaccggtttgcatgctcaatctttataagtccttttataaatttc

SEQ ID NO: 21 - DNA, 25 bp, Artificial Sequence
  PCR primer
gtatgcttggttgatgataatcggg

SEQ ID NO: 22 - DNA, 24 bp, Artificial Sequence
  PCR primer
gcagtcctatccctttcaactcgc

SEQ ID NO: 23 - DNA, 26 bp, Artificial Sequence
  PCR primer
cgaaattgcgggtattcaagttggag

SEQ ID NO: 24 - DNA, 26 bp, Artificial Sequence
  PCR primer
gcatttttcgcaccgttagttgttgg

SEQ ID NO: 25 - DNA, 36 bp, Artificial Sequence
  PCR primer
aaccggtttcccgggaagacaaacgagagtagaaac

SEQ ID NO: 26 - DNA, 39 bp, Artificial Sequence
  PCR primer
aaccggtttggatcccgtagcatctgcatgcggtgtacg

SEQ ID NO: 27 - DNA, 36 bp, Artificial Sequence
  PCR primer
aaccggtttgagctcgcgaactgcttctctcattac

SEQ ID NO: 28 - DNA, 46 bp, Artificial Sequence
  PCR primer
ttcccgggacgtacggtaccttaagtgaacgcaaaggttagcaaac

SEQ ID NO: 29 - DNA, 38 bp, Artificial Sequence
  PCR primer
aaccggtttggatccactgctcctcctttctgattatg

SEQ ID NO: 30 - DNA, 36 bp, Artificial Sequence
  PCR primer
aaccggtttcccgggcacacctcttccagaaagtac

SEQ ID NO: 31 - DNA, 38 bp, Artificial Sequence
  PCR primer
aaccggttttctagagctacagctgcatctgactcttc

SEQ ID NO: 32 - DNA, 38 bp, Artificial Sequence
  PCR primer
aaccggtttggatccccaaaaatcgagcaaaattataa

SEQ ID NO: 33 - DNA, 42 bp, Artificial Sequence
  PCR primer
gcactcgttatcaaaaagaaagtacagtatacattcataagg

SEQ ID NO: 34 - DNA, 35 bp, Artificial Sequence
  PCR primer
gctttcttacgcctgtacgatcgatcttcatcagc

SEQ ID NO: 35 - DNA, 30 bp, Artificial Sequence
  PCR primer
gtgccacctgacccgcgggtctaagaaacc

SEQ ID NO: 36 - DNA, 32 bp, Artificial Sequence
  PCR primer
gaaccctgttacatgctagctcattacacttc

SEQ ID NO: 37 - DNA, 48 bp, Artificial Sequence
  PCR primer
gcatcgccagtcactatggcgtgctgcaagcgctatatgcgttgatgc

SEQ ID NO: 38 - DNA, 36 bp, Artificial Sequence
  PCR primer
caaagggggaaatgtacattcgaagatctccggagc

SEQ ID NO: 39 - DNA, 30 bp, Artificial Sequence; identical to SEQ ID NO: 35
  PCR primer

SEQ ID NO: 40 - DNA, 31 bp, Artificial Sequence
  PCR primer
gcgtatcacgaggccgcggttaacggaacgc

SEQ ID NO: 41 - DNA, 31 bp, Artificial Sequence
  PCR primer
gcgttccgttaaccgcggcctcgtgatacgc

SEQ ID NO: 42 - DNA, 30 bp, Artificial Sequence; identical to SEQ ID NO: 35
  PCR primer

SEQ ID NO: 43 - DNA, 32 bp, Artificial Sequence; identical to SEQ ID NO: 36
  PCR primer

SEQ ID NO: 44 - DNA, 48 bp, Artificial Sequence; identical to SEQ ID NO: 37
  PCR primer

SEQ ID NO: 45 - DNA, 9909 bp, Artificial Sequence
  P306pMM1522+tet
aatgtacaatggtccaatgtacattcgaagatctccggagctcccgggatccggtaccggccgcatgccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtcttttgtactttacgcggtaccaccctcgtttgtaatcgtaatgattaccttatgaatgtatactgtagtttctttttgataacgagtgctttgcagggtgacactctgaggtgagttcattttgagttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagtcaaccactccgcgggtctaagaaaccattattatcatgacattaacctataaaaataggcgtatcacgaggccctttcgtcttcaagaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaaggggga

SEQ ID NO: 46 - DNA, 9771 bp, Artificial Sequence
  P503pMM1522+tet
aatgtacattcgaagatctccggagctcccgggatccggtaccggccgcatgccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtctttttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagccaaccactccgcgggtctaagaaaccattattatcatgacattaacctataaaaataggcgtatcacgaggccctttcgtcttcaagaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaaggggga

SEQ ID NO: 47 - DNA, 9920 bp, Artificial Sequence
  P306pHIS1522+tet
ccggcctgagaggatcgcatcaccatcaccatcactaaccggtccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggagtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgcaaagcactcgttatcaaaaagaaactacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacgatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccgcgggtctaagaaaccattattatcatgacattaacctataaaaataggcgtatcacgaggccctttcgtcttcaagaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgtacaatggtccaaactagttcgaagatctccggagctcccgggatccggtaccggccgcatg

SEQ ID NO: 48 - DNA, 9797 bp, Artificial Sequence
  P503pHIS1522+tet
ccggcctgagaggatcgcatcaccatcaccatcactaaccggtccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtctttttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagccaaccactccgcgggtctaagaaaccattattatcatgacattaacctataaaaataggcgtatcacgaggccctttcgtcttcaagaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgtacaatggtccaaactagttcgaagatctccggagctcccgggatccggtaccggccgcatg

SEQ ID NO: 49 - DNA, 8936 bp, Artificial Sequence
  P503pSTOP+tet
aaggaggtgaatgtacaatggtccaaactagttcgaagatctccggagctcccgggatccggtaccggccgcatgccggctaaacctcgcgaacggattcaccggtccaagaattggagctaattaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtctttttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagtcaaccactccgcggccgcgggtctaagaaaccattattatcatgacattaacctataaaaataggcgtatcacgaggccctttcgtcttcaagaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgacataaacatttaattaatatatgatgagataaagttagtttattggataaacaaactaactcaattaagatagttgatggataaacttgttcacttaaatc

SEQ ID NO: 50 - DNA, 8194 bp, Artificial Sequence
  P503pMM1522-tet
gaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagaatgtacaatggtccaaactagttcgaagatctccggagctcccgggatccggtaccggccgcatgccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggagtggttggctatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgttatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacgatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccggctagctcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccatcaccctggatgctgtaggcataggcttggttatgccggtactgccgggcctcttgcgggatatcgtccattccgacagcatcgccagtcactatggcgtgctgcaagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaaggggga

SEQ ID NO: 51 - DNA, 8317 bp, Artificial Sequence
  P306pMM1522-tet
aatgtacaatggtccaaactagttcgaagatctccggagctcccgggatccggtaccggccgcatgccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacccgcggagtggttgactatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgcaaagcactcgttatcaaaaagaaactacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgtaatgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataaggaggagaacacagatgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtagtatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaaccaaaaatcgagcaaaattataattttgctcgatttttttgcgttccgttaaccggctagctcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccatcaccctggatgctgtaggcataggcttggttatgccggtactgccgggcctcttgcgggatatcgtccattccgacagcatcgccagtcactatggcgtgctgcaagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaaggggga

SEQ ID NO: 52 - DNA, 8312 bp, Artificial Sequence
  P503pHIS1522-tet
ccggcctgagaggatcgcatcaccatcaccatcactaaccggtccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttgctagccggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtctttttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagccaaccactccgcgggggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgcaagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgtacaatggtccaaactagttcgaagatctccggagctcccgggatccggtaccggccgcatg

SEQ ID NO: 53 - DNA, 8435 bp, Artificial Sequence
  P306pHIS1522-tet
ccggcctgagaggatcgcatcaccatcaccatcactaaccggtccaagaattggagccaatcaattcttgcggagaactgtgaatgcgcaaaccaacccttggcagaacatatccatcgcgtccgccatctccagcagccgcacgcggcgcatctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttgctagccggttaacggaacgcaaaaaaatcgagcaaaattataattttgctcgatttttggttaatatcttaccgtttttgcttcataagcagaaatgtcgtcttcaaaagaaagcgtatagccaatttcatcatatccgttaacaagcatatatttccaatggccattgatatcaaacgtacgttcaaaacctgcttcatcggtaattttttgctgatgaagatcgatcgtacaggcgtaagaagcactttgagactctttcattaaataggaaacatcttcttttggtaaaacaataggtaaaataccattttttagacagttttgtttaaaaatatccgcaaatgaaggtgcgataatagcacgaaacccgtagtctaaaagcgcccatggcgcgtgttcacgtgacgaaccacatccgaagttctcgtcagctacaagaattgttgctccttgattttgaggctgatttaattcaaacgtttcatttaacgtctcatcttcattatatcgccaatcaaaaaatagaaattggccaaagccagtacgctcaattcttttcaaaaattgctttgggataatttgatcagtatctacgtttgctcggtcgagacctgctactttccctgtatggattgtaaaagcattcatctgtgttctcctccttatcgcaccgcttgttcttgtagtttgcgtacatcaacaaaatgtccatataccgctgccgctgctgccattaccgggcttactaaatgcgttctggcgccttttccttggcgtccttcaaaattccggttagaagtggacgcacagtgttcgccttgtggaactaagtcagagttcatgcctagacacatgctgcagcctgagtctctccaatcaaaacctgcttcaataaaaatagcagataacccttcttcttcagccactcgctttactttttgagaacctggtacaatgattgctcgcacatctggtgacactttctttcctttaataaaggctgccacgtcacgaagatcagacaaacgagcattcgtacatgaaccgataaatacatgctgaactggaatctcttctattgccatgccaggctgtaagcccatatactcaagtgctcgtgacaaagactttctctcagcttctgttttacactcttctaatgtaggaacttgcttgctaacataagaactcatcgccgggtttgttccccatgttacaacaggttcaattgtatctgcatcaatttccagcgtttcatcgtacgttgcaccttcatctgttgctaaagctgcccaagcttccactgctttttcaaactgttctcctttaggagcatactgacgtcctttaatataagagaacgtgacatcgtctggactgattaaaccagcttttgcaccggcttcaatagacatattacaaatggtcatgcgctcttccattgaaagatcgcgaatacattcgcccgtaaattctacgattgaacccgttccaaaattcacgccgaatttttgaattattgctaacacaatgtccttagcataaacgccaggctgctttttgccattcactttcacttgtagcgtttttggtttcgcttgccatagcgtttgtgttgctaaaacatgctctacctcactagttcctatcccaaaagcaagcgctccaaatgcaccgtgagtagatgtatgactatctccgcacacaatggtttttccaggctgagttaatcctaattccggtccaatgatatgcacaattccttgctcttcacttgttaaatctaaaagcttcacaccaaactcatgacagttttcagatagtttgtctacttgtttctttgcaatcggatctttaatattaaaacggtcaactgttggaatgttgtgatccattgtagcaaacgttaagtctggacgcttaacttggcgatttgccacgcgaagaccttcaaatgcttgaggagacgttacttcatgaataagatgaagatcgatatataataagtccggcttcccctcttctcgacgtacaacatgttgttcccaaagcttatcaataattgtttttggtttcattacgtattttctcatcctttcgttttctagataaaagaaaaaagacttctcacccctaaatagaccctatcaaatgatataggtcaaggggcgaaaagtcttttgtactttacgcggtaccaccctcgtttgtaatcgtaatgattaccttatgaatgtatactgtagtttctttttgataacgagtgctttgcagggtgacactctgaggtgagttcattttgagttggtaacgtcggcttccagcaacccgactctctgttgatacctaaacatcaaaactacttatcctcttcaacgttttaaaacgctatatgctttttgaacaatgctttacacatcatattttaaattaattacaattaattaaaatttttagaaaacttatatttgaggactatcttacgcccatttctatgcatagtcaaccactccgcgggggttaatgatacgcttccctcttttaattgaaccctgttacattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgcaagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctgacgcgtgtaactgcgaaggtaaaacgggtgattgaaaataaactaacaaatgaagaggaacgaaagagaagtctagaatttgttacgtttatatcggatgaattactgcaaaatgatacggagcctcgcacatttattattcagaactctcttttatcgctagagaaaatccatactctcgcacagaccaaagaggtagaggaatataaagaagtcataactactctgacaagacatgattcataatttgaaaagcaggtaaactaacccaaaaggcaagactctccgtagtttagaaaagagcaggttgctaataacatataaacagccagttgccgttatgataggtgactggctgcatgggatgaaaaggtgagggtggagacagacataacactcttaatagaagagggtaattctttctcttttatagaaaatcaattaattgaaagtagctccttcattcttaagatcaaacgtgatatagtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagtcttctcgagaataatatttccttctaagtcggttagaattccgttaagatagtcgactcctatatcaataccaatcgagtagcctgcattcttattaaaaacaagcattacaggtcttctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgtacaatggtccaaactagttcgaagatctccggagctcccgggatccggtaccggccgcatg

SEQ ID NO: 54 - DNA, 789 bp, Bacillus megaterium
ttgaaaaaaattaaagtatgcttggttgatgataatcgggaactaattggtttgcttgaagattatatttcagagcaagaagacatggaagtaattggagtagcttacaatggacaagagtgtttatccattttaaaagataaaaatccggatgttcttgtattagacattattatgccgcatctagatggattagcagttttaggtcagttaaaagaaatgaaaaaagaaaattatccaaatgtcattatgctaacagcatttggtcaagaagacgtaacgaaaaaagcggtggatttaggagctgcttattttattctaaaaccgtttgatatggaaaacttagtgaatcatattcgtcaagttagcggacaaacatcttcatttatgcaacgttcatcttcttccatgcgttcacatcgcgacagcaaaccagcaaatttagatgcaagcattacgagcatcattcacgaaattggtgtacctgctcatatcaaaggctatttgtatttgagagaagccatttcaatggtatataaggatattgagctgcttgggtcgattacaaaagtactgtatcctgatattgcaaaaaaatataatacaacggcaagccgcgttgagcgcgccattcgtcatgccattgaagtagcatggagccgaggaaacattgattccatttcttcattattcggatatactgtcagcatgtcaaaagcgaagccgacaaatagtgaatttatcgccatggttgcggataaattacgccttgaacataaagcgagttga

SEQ ID NO: 55 - DNA, 1338 bp, Bacillus megaterium
atggtccaaactagtactaataaaattaatcattttgaaagcgcaaacaaagttttatacgaaggtaaagattctaaaaatcctttagcttttaaatactataaccctgaagaagtagtaggcggtaaaacgatgaaagatcagctgcgtttttctgttgcttactggcacacgtttacagcagatggtacggatccttttggcgcagctactatgcaaagatcttgggatagatatgatggaatggatttagcaaaagcgagagttgaggcagcttttcaactttttgaaacattaaatgtgccattcttcgcatttcatgatcgagacattgctccggagggcagtacgttacaagaaacaaataaaaatcttgatgttatcgtcactatgatcaaagaatacatgcaaacaagtaacgtaaagttattatggaacacagcgaatatgtttacaaatccccggttcgttcatggtgcagctacttcttgtaatgcagatgtatttgcttatgcggcggctcaagtaaaaaaggggcttgaaacagcaaaagaactaggcgcagaaaattatgtattctggggcggccgtgagggatatgaaacactgcttaacacaaatttacagttagagttagataacttagccagatttatgcatatggctgtagattacgcaaccgaaattggatatacggggcagtttttaattgagcctaaacctaaagagcctacgacacatcagtatgataccgatgcagcaacaactatttcatttttaagacaatacggattagataaatacttcaaacttaatctagaagctaaccacgcaacgttagctgggcatacatttgaacacgaattacgagtagctagagtacaaggcttattaggctcagtggatgcaaaccaaggtgaccctcttttaggctgggatacagatgaattcccaacggatttatattctacaacgctagcaatgtatgaaattctgcaaaatggagggctgggcagcggaggattaaactttgatgctaaagtacgcagaggttcttttgaacaagacgatttactctatgctcatgtagcaggaatggatgcgtttgctcgaggattgaaagtagcacataaattagtagaagatcgtgttttcgaaaatgttattaacgagcgctacagcagctttaaagaaggaattgggcttgagattgtggaaggaaaagctaacttccatacactggagcagtacgcgtttaaaaatccgaatattgcgaataaatcaggaagacaagaacgtttaaagtccattttaaatcaatatattttagaagtttaa

SEQ ID NO: 56 - DNA, 1167 bp, Bacillus megaterium
gtggttattattcaaattgcagatcaagctttagtaaaaaaaatgaatcaaaaattaatattagatgaaattttgaagaactcccctgtctccagggcaactctctctgagattacaggattaaacaagtctactgtctcctctcaagtaaatacactgcttgaaaaagattttatttttgaaattggggcagggcaatctagaggcggcagaagacctgtaatgcttgtttttaataagaatgcaggctactcgattggtattgatataggagtcgactatcttaacggaattctaaccgacttagaaggaaatattattctcgagaagacttctgacttgtctagttcttccgctagtgaagtaaaagagattttatttgcacttattcatggttttgtaacccatatgcctgagtccccttatggtctagtcggaataggaatttgtgttccaggccttgtagatcgtcatcagcaaattattttcatgcctaacttaaattggaatatcaaagatttgcagtttttaattgagagtgagtttaatgttccggtttttgttgaaaatgaagctaatgcaggagcatacggtgaaaaagtatttggtatgacaaaaaactatgaaaacatcgtttacatcagtattaatatcggaattggaactggacttgttattaacaacgaattgtataaaggtgttcagggtttttctggggaaatgggtcatatgacgatagattttaatggacccaaatgcagctgtggaaatcgaggctgttgggaattatatgcttctgaaaaagcgttactggcttcgctctctaaagaagaaaagaatatttctcgaaaagagattgtggaacgcgcaaataaaaatgatgtagaaatgttaaatgcacttcaaaactttggcttttatatcggaattggattaaccaatatccttaatacatttgatatagaagctgttatcttgagaaatcatataattgaatctcatcccattgttttaaatacgattaaaaacgaagtttcttctagagtccattctcatttagacaataaatgtgaactattgccttcttcgttaggaaaaaatgcacctgctttaggagcggtttctatcgttattgattcttttttaagtgttacccctataagttag

SEQ ID NO: 57 - DNA, 1416 bp, Bacillus megaterium
atgaaaccaaaaacaattattgataagctttgggaacaacatgttgtacgtcgagaagaggggaagccggacttattatatatcgatcttcatcttattcatgaagtaacgtctcctcaagcatttgaaggtcttcgcgtggcaaatcgccaagttaagcgtccagacttaacgtttgctacaatggatcacaacattccaacagttgaccgttttaatattaaagatccgattgcaaagaaacaagtagacaaactatctgaaaactgtcatgagtttggtgtgaagcttttagatttaacaagtgaagagcaaggaattgtgcatatcattggaccggaattaggattaactcagcctggaaaaaccattgtgtgcggagatagtcatacatctactcacggtgcatttggagcgcttgcttttgggataggaactagtgaggtagagcatgttttagcaacacaaacgctatggcaagcgaaaccaaaaacgctacaagtgaaagtgaatggcaaaaagcagcctggcgtttatgctaaggacattgtgttagcaataattcaaaaattcggcgtgaattttggaacgggttcaatcgtagaatttacgggcgaatgtattcgcgatctttcaatggaagagcgcatgaccatttgtaatatgtctattgaagccggtgcaaaagctggtttaatcagtccagacgatgtcacgttctcttatattaaaggacgtcagtatgctcctaaaggagaacagtttgaaaaagcagtggaagcttgggcagctttagcaacagatgaaggtgcaacgtacgatgaaacgctggaaattgatgcagatacaattgaacctgttgtaacatggggaacaaacccggcgatgagttcttatgttagcaagcaagttcctacattagaagagtgtaaaacagaagctgagagaaagtctttgtcacgagcacttgagtatatgggcttacagcctggcatggcaatagaagagattccagttcagcatgtatttatcggttcatgtacgaatgctcgtttgtctgatcttcgtgacgtggcagcctttattaaaggaaagaaagtgtcaccagatgtgcgagcaatcattgtaccaggttctcaaaaagtaaagcgagtggctgaagaagaagggttatctgctatttttattgaagcaggttttgattggagagactcaggctgcagcatgtgtctaggcatgaactctgacttagttccacaaggcgaacactgtgcgtccacttctaaccggaattttgaaggacgccaaggaaaaggcgccagaacgcatttagtaagcccggtaatggcagcagcggcagcggtatatggacattttgttgatgtacgcaaactacaagaacaagcggtgcgataa

SEQ ID NO: 58 - DNA, 591 bp, Bacillus megaterium
atgaatgcttttacaatccatacagggaaagtagcaggtctcgaccgagcaaacgtagatactgatcaaattatcccaaagcaatttttgaaaagaattgagcgtactggctttggccaatttctattttttgattggcgatataatgaagatgagacgttaaatgaaacgtttgaattaaatcagcctcaaaatcaaggagcaacaattcttgtagctgacgagaacttcggatgtggttcgtcacgtgaacacgcgccatgggcgcttttagactacgggtttcgtgctattatcgcaccttcatttgcggatatttttaaacaaaactgtctaaaaaatggtattttacctattgttttaccaaaagaagatgtttcctatttaatgaaagagtctcaaagtgcttcttacgcctgtacaatcgatcttcatcagcaaaaaattaccgatgaagcaggttttgaacgtacgtttgatatcaatggccattggaaatatatgcttgttaacggatatgatgaaattggctatacgctttcttttgaagacgacatttctgcttatgaagcaaaaacggtaagatattaa

SEQ ID NO: 59 - DNA, 469 bp, Artificial Sequence
  Pleu-1 Sequence
ccgcggagtggttggctatgcatagaaatgggcgtaagatagtcctcaaatataagttttctaaaaattttaattaattgtaattaatttaaaatatgatgtgtaaagcattgttcaaaaagcatatagcgttttaaaacgttgaagaggataagtagttttgatgtttaggtatcaacagagagtcgggttgctggaagccgacgttaccaactcaaaatgaactcacctcagagtgtcaccctgaaattcagtaagggtgaccgagtgtacttccaaagcactcgttatcaaaaagaatgtacagtatacattcataaggtaatcattacgattacaaacgagggtggtaccgcgtaaagtacaaaagacttttcgccccttgacctatatcatttgatagggtctatttaggggtgagaagtcttttttcttttatctagaaaacgaaaggatgagaaaatacgta

SEQ ID NO: 60 - protein, 470 aa, Bacillus megaterium
KPKTIIDKLWEQHVVRREEGKPDLLYIDLHLIHEVTSPQAFEGLRVANRQVKRPDLTFATMDHNIPTVDRFNIKDPIAKKQVDKLSENCHEFGVKLLDLTSEEQGIVHIIGPELGLTQPGKTIVCGDSHTSTHGAFGALAFGIGTSEVEHVLATQTLWQAKPKTLQVKVNGKKQPGVYAKDIVLAIIQKFGVNFGTGSIVEFTGECIRDLSMEERMTICNMSIEAGAKAGLISPDDVTFSYIKGRQYAPKGEQFEKAVEAWAALATDEGATYDETLEIDADTIEPVVTWGTNPAMSSYVSKQVPTLEECKTEAERKSLSRALEYMGLQPGMAIEEIPVQHVFIGSCTNARLSDLRDVAAFIKGKKVSPDVRAIIVPGSQKVKRVAEEEGLSAIFIEAGFDWRDSGCSMCLGMNSDLVPQGEHCASTSNRNFEGRQGKGARTHLVSPVMAAAAAVYGHFVDVRKLQEQAVR

SEQ ID NO: 61 - protein, 196 aa, Bacillus Megaterium
MNAFTIHTGKVAGLDRANVDTDQIIPKQFLKRIERTGFGQFLFFDWRYNEDETLNETFELNQPQNQGATILVADENFGCGSSREHAPWALLDYGFRAIIAPSFADIFKQNCLKNGILPIVLPKEDVSYLMKESQSASYACTIDLHQQKITDEAGFERTFDINGHWKYMLVNGYDEIGYTLSFEDDISAYEAKTVRY

(19) United States
(12) Patent Application Publication
(10) Pub. No.: US 2017/0335360 A1
(43) Pub. Date: Nov. 23, 2017

(54) BACILLUS MEGATERIUM RECOMBINANT PROTEIN EXPRESSION SYSTEM

(71) Applicant: Merck Sharp & Dohme Corp., Rahway, NJ (US)

(72) Inventors: Susan L. Secore, Telford, PA (US); Jean-Luc Bodmer, Concord, MA (US); Jon H. Heinrichs, Doylestown, PA (US); Johannes C. Scholten, San Diego, CA (US)

(73) Assignee: Merck Sharp & Dohme Corp., Rahway, NJ (US)

(21) Appl. No.: 15/522,593

(22) PCT Filed: Oct. 26, 2015
(86) PCT No.: PCT/US15/57285

§ 371 Date: Apr. 27, 2017

Related U.S. Application Data
(60) Provisional application No. 62/072,652, filed on Oct. 30, 2014.

(51) Int. Cl. C12P 21/00; C12N 9/90; C12N 9/88; C12N 9/10; C12N 15/75; C07K 14/32

(52) U.S. Cl. CPC C12P 21/00; C12Y 503/01005; C12Y 402/01033; C12N 15/75; C07K 14/32; C12N 9/88; C12N 9/1051; C12N 9/90

(57) ABSTRACT

The present invention relates to isolated or purified asporogenous Bacillus megaterium (B. megaterium) strains comprising a B. megaterium genome, wherein said genome is modified in that the spo0A gene is deleted or functionally deleted and the strain does not produce spores. The aspororogenous strains of the invention may be further modified by a deletion or functional deletion of one or more genes selected from xylA, xylR, leuC and leuD. The strains of the invention may further comprise an expression vector, wherein the expression vector comprises a sequence of nucleotides that encodes a heterologous polypeptide, operatively liked to a promoter. Also provided by the invention are modified expression vectors and promoters for use in the B. megaterium expression systems of the invention and methods of use thereof.

### CROSS-REFERENCE TO RELATED APPLICATIONS

This application claims the benefit of U.S. Provisional Application No. 62/072,652, filed Oct. 30, 2014, the contents of which are hereby incorporated by reference in their entirety.

### REFERENCE TO SEQUENCE LISTING SUBMITTED ELECTRONICALLY

The sequence listing of the present application is submitted electronically via EFS-Web as an ASCII formatted sequence listing with a file name “23789WOPCTSEQ.TXT”, creation date of Oct. 16, 2015, and a size of 155 KB. This sequence listing submitted via EFS-Web is part of the specification and is herein incorporated by reference in its entirety.

### BACKGROUND OF THE INVENTION

Bacillus megaterium are large, gram positive, sporulating bacteria that are able to utilize a wide variety of carbon including xylose as a sole carbon source. Bacillus megaterium (B. megaterium) has been shown to be able to stably express proteins both intra- and extra-cellularly. B. megaterium is an important species because of its production of natural products such as α-amylases, β-amylases, glucose dehydrogenase, glutamate, vitamin B12, and penicillin amidase (Vary, P., Prime time for Bacillus megaterium. Microbiol. 140:1001-1013 (1994)). There are several advantages associated with use of B. megaterium for recombinant expression of heterologous proteins, including the fact that B. megaterium expresses only one secreted protease and no alkaline proteases, and does not express endotoxin. These features allow cloning and expression of heterologous proteins without degradation. Additionally, plasmids can be stably maintained in this system.

A B. megaterium protein expression system has been successfully used to express a variety of proteins including penicillin G amidase (Yang et al. Microb. Cell Fact. 5:36 (2006); Hollmann et al. Eng. Life Sci. 6: 470-474 (2006)), Clostridium difficile toxins A and B (Yang et al. BMC Microbiology, 8:192 (2008); Burger et al. Biochem. Biophys. Res. Commun. 307:584-588 (2003)), Dextransucrase (Malten et al. Biotechnol Bioeng. 89(2): 206-218 (2005); Hollmann et al, (2006), supra), glucose dehydrogenase (Meinhardt et al. Appl. Microbiol. Biotechnol. 30: 343-350 (1989)), functional scFv antibody fragments (Jordan et al. Microbial Cell Factories 6:2 (2007)), and Levansucrose (Malten et al. Appl. Environ. Microbiol., 72(2): 1677-1679 (2006); Hollmann et al. (2006), supra).

Despite the advantages of using B. megaterium as a component of a protein expression system, known B. megaterium expression systems are not optimal for use as manufacturing platforms for recombinant protein production because of the requirement for the use of antibiotics for selection and maintenance of the expression plasmids and the difficulties associated with working with a sporulating bacterium as a cell substrate. Additionally, known expression systems utilize B. megaterium substrates that metabolize xylose, which is used to induce expression, making it difficult to maintain a steady state during fermentations. Thus, there is a need for an improved B megaterium expression system that is amenable for use as a commercial protein manufacturing platform.

### SUMMARY OF THE INVENTION

The invention is related to an isolated or purified asporogenous Bacillus megaterium (B. megaterium) strain comprising a B. megaterium genome, wherein said genome is modified in that the spo0A gene is deleted or functionally deleted, wherein the strain does not produce spores. In embodiments of the invention, the B. megaterium genome is further modified by a deletion or functional deletion of the xylA and xylR genes, wherein the strain is unable to metabolize xylose. In additional embodiments, the B. megaterium genome further comprises a deletion or functional deletion of a gene that functions as part of the ilv-leu operon, wherein the strain is leucine auxotrophic. In specific embodiments, the B. megaterium genome comprises a deletion or functional deletion of the leuC and leuD genes.

The invention also relates to an expression vector, comprising the leuC and leuD genes and a sequence of nucleotides that encodes a heterologous polypeptide, operatively liked to a promoter. In embodiments of this aspect of the invention, the leuC and leuD genes comprise the nucleotide sequences set forth in SEQ ID NO:57 and SEQ ID NO:58. Also provided by the invention is an asporogenous B. megaterium strain of the invention, further comprising said expression vector.

The invention further relates to an Ilv-leu variant promoter selected from the group consisting of: P503, P306, P201 and Pleu-1. In embodiments of this aspect of the invention, the promoter comprises a sequence of nucleotides as set forth in SEQ ID NO:7, SEQ ID NO:8, SEQ ID NO:9, or SEQ ID NO:59. In further embodiments, the promoter comprises a sequence of nucleotides as set forth in SEQ ID NO:7, SEQ ID NO:8, or SEQ ID NO:9. Also provided by the invention are expression vectors comprising the leuC and leuD genes, wherein the leuC and leuD genes are operatively linked to an Ilv-leu variant promoter of the invention.

A further aspect of the invention is a method of producing a heterologous protein in a B. megaterium host cell comprising: (a) transforming a modified B. megaterium cell with an expression vector comprising a sequence of nucleotide that encodes the heterologous protein, wherein the modified cell comprises a B. megaterium genome that has been modified in that the spo0A gene is deleted or functionally deleted and wherein the modified cell does not produce spores; (b) cultivating the transformed modified B. megaterium cell under conditions that permit expression of the nucleotide sequence to produce the heterologous protein; and (c) optionally isolating the heterologous protein. In embodiments of this aspect of the invention, the B. megaterium genome comprises a deletion or functional deletion of the xylA and xylR genes and cannot metabolize xylose. In further embodiments, the B. megaterium genome comprises a deletion or functional deletion of the leuC and leuD genes and the expression vector comprises a sequence of nucleotides that encodes leuC and a sequence of nucleotides that encodes leuD. In additional embodiments, the leuC and leuD genes are operably linked to an ilv-leu promoter, or a variant ilv-leu promoter selected from the grup consisting of: P503, P306, P201 and Pleu-1.

As used throughout the specification and in the appended claims, the singular forms “a,” “an,” and “the” include the plural reference unless the context clearly dictates otherwise.

As used throughout the specification and appended claims, the following definitions and abbreviations apply:

A “functional deletion” of a particular gene occurs when the production or function of that gene product is genetically knocked-out. The phrase “functional deletion” as used herein broadly encompasses modifications to the gene or to a sequence controlling transcription of the gene that have the effect of rendering a particular gene product nonfunctional. Generally speaking, functional deletions take the form of a partial or total deletion of a gene. However, one of skill in the art will readily acknowledge that other manipulations, including but not limited to making a modification which introduces a frame shift mutation, such as through an insertion or small deletion, will also achieve a functional deletion. A functional deletion also encompasses the modification of a gene through the introduction of one or more point mutations or the insertion of one or more nucleotides within a nucleotide sequence of a gene.

As used herein, the term “recombinant” refers to a polypeptide or nucleic acid that does not exist in nature. The term “recombinant” polypeptide refers to a polypeptide that is prepared, expressed, created, or isolated by recombinant means, such as polypeptides expressed using a recombinant expression vector transfected into a host cell. A recombinant polynucleotide may also include two or more nucleotide sequences artificially combined and present together in a longer polynucleotide sequence, wherein the two sequences are not found together (e.g. attached or fused) in nature, e.g. a promoter and a heterologous nucleotide sequence encoding a polypeptide that are normally not found together in nature or a vector and a heterologous nucleotide sequence.

As used herein, the terms “isolated” or “purified” refer to a molecule (e.g., nucleic acid, polypeptide, bacterial strain, etc.) that is at least partially separated from other molecules normally associated with it in its native state. An “isolated or purified polypeptide” is substantially free of other biological molecules naturally associated with the polypeptide such as nucleic acids, proteins, lipids, carbohydrates, cellular debris and growth media. An “isolated or purified nucleic acid” is at least partially separated from nucleic acids which normally flank the polynucleotide in its native state. Thus, polynucleotides fused to regulatory or coding sequences with which they are not normally associated, for example as the result of recombinant techniques, are considered isolated herein. Such molecules are considered isolated even when present, for example in the chromosome of a host cell, or in a nucleic acid solution. Generally, the terms “isolated” and “purified” are not intended to refer to a complete absence of such material or to an absence of water, buffers, or salts, unless they are present in amounts that substantially interfere with experimental or therapeutic use of the molecule.

As used herein, “homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

The term “cassette” refers to a nucleic acid molecule which comprises at least one nucleic acid sequence that is to be expressed, along with its transcription and translational control sequences. Changing the cassette, will cause the vector into which is incorporated to direct the expression of different sequence or combination of sequences. In the context of the present invention, the nucleic acid sequences present in the cassette will usually encode any polypeptide of interest such as an immunogen. Because of the restriction sites engineered to be present at the 5′ and 3′ ends, the cassette can be easily inserted, removed or replaced with another cassette.

The term “promoter” refers to a recognition site on a DNA strand to which an RNA polymerase binds. The promoter forms an initiation complex with RNA polymerase to initiate and drive transcriptional activity. The complex can be modified by activating sequences such as enhancers, or inhibiting sequences such as silencers.

As used herein, the term “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering or substantially altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “leuC” and “leuD” refer to the large and small subunits of 3-isopropylmalate dehydratase (or isomerase), respectively. Prior to 2001, Bacillus subtilis nomenclature specified that leuC was the 3-isopropylmalate dehydrogenase gene and E. coli nomenclature specified that leuC was the large subunit of 3-isopropylmalate dehydratase gene (Microbiology 147:1-2 (2001)). At this time, Bacillus megaterium followed the nomenclature of Bacillus subtilis and literature referring to leuC in B. megaterium was generally referring to the 3-isopropylmalate dehydrogenase gene. After 2001, the B. subtilis nomenclature was standardized to the E. coli nomenclature and leuC in B. megaterium began to refer to 3-isopropylmalate dehydratase, as used herin.

The following abbreviations and gene designations are used herein and have the following meanings: aac-aphD=aminoglycoside resistance determinant of the transposon Tn4001, gentamicin/kanamycin resistance marker; bp=base pairs; dfra=Staphylococcus aureus constitutive promoter; CDTa=binary toxin A from C. difficile; kb=kilobase; FACS=flourescence activated cell sorting; leuC=3-isopropylmalate dehydratase gene, large subunit; leuD=3-isopropylmalate dehydratase gene, small subunit; ORI=origin of replication; leuCD=leuC+leuD genes; PCR=polymerase chain reaction; Pdfra=dihydrofolate reductase promoter; spo0A=stage 0 sporulation protein A; Pleu=ilv-leu wild type promoter (alternatively called pleuleu); Pleu-1=variant ilv-leu promoter with single point mutation; P201=B. megaterium ilv-leu variant promoter (alternatively called pleu201); P306=B. megaterium ilv-leu variant promoter (alternatively called pleu306); P503=B. megaterium ilv-leu variant promoter (alternatively called pleu503); TcdB=toxin B from Clostridum difficile; TSA=tryptic soy agar; xylA=xylose isomerase gene; and xylR=xylose repressor gene.

### BRIEF DESCRIPTION OF THE DRAWINGS

FIG. 1 provides the nucleotide sequence of the resistance marker aac-aphD under the control of Pdfra (SEQ ID NO:1, see Example 2). The P-drfA sequence is underlined. A BamHI resitriction site in shown in bold italics.

FIG. 2 provides a map of the integration vector pCL10, which was originally described for gene inactivation in Staphylococcus aureus. It contains a temperature sensitive lethal mutation in the gram+ ORI. See Example 2.

FIG. 3 provides the nucleotide sequence of the spo0A knockout cassette, which comprises regions 5′ and 3′ of spo0A plus PdfrA aacA-aphD (SEQ ID NO:2, see Example 2). The location of the PdfrA aacA-aphD resistance marker is underlined. A SphI restriction site, used to add the resistance marker to the construct, is shown in bold, italics.

FIG. 4 shows the possible integration events resulting from the integration protocol used to knock-out the spoOA gene, as described in Example 3. Pictured in FIG. 4A is B. megaterium chromosomal DNA with the spoOA and flanking regions indicated. Below the chromosomal DNA is the pCL10 integration vector, in which the aacA-aphD resistance marker flanked by the upstream and downstream regions of spoOA have been inserted. FIG. 4B shows the B. megaterium chromosomal DNA following a single cross-over event, which leads to integration of the entire plasmid and maintenance of kanamycin and chloramphenicol resistance. The bacteria maintains a functional copy of spoOA. FIG. 4C shows B. megaterium chromosomal DNA following a double cross-over event, which leads to the integration of only the aacA-aphD marker and flanking regions. In the case of a double cross-over, kanamycin resistance is maintained whereas chloramphenicol resistance is lost. In this event, spoOA is removed from the chromosomal DNA.

FIG. 5 shows results from a PCR amplification of selected colonies following the integration protocol related to spoOA (see Example 3). Oligonucelotide primers were designed to amplify a 788 bp fragment of the spoOA gene from chromosomal DNA isolated from four colonies that met the desired antibiotic resistance profile (lanes 2-5, marked “ΔspoOA 1−ΔspoOA 4”). Also shown is a positive control (lane 1, DSM319 genomic DNA) and a single cross transformant (lane 6). Note that the spoOA PCR product is not visible in lanes 2 and 3, confirming a double cross-over (and thus knock-out of spoOA) for the ΔspoOA 1 and ΔspoOA 2 isolates.

FIG. 6 shows results from a PCR amplification of DNA from the ΔspoOA 1−ΔspoOA 4 isolates (lanes 3-6) using oligonucleotide primers designed to target the SpoOA flanking region. Also shown are amplification products resulting from PCR of wild-type genomic DNA (lane 2, DSM319) and a single cross transformant (lane 7). PCR products were run on a 1% agarose gel. The expected size of PCR fragments was 3.1 kb for the wild-type nucleotide sequence (i.e. no cross-over) and 3.8 kb for ΔspoOA (i.e. double cross-over).

FIG. 7 shows expression of nap_3 mTcdB in asporogenous B. megaterium strain spoOA-A. Shown in FIG. 7A are samples run on a polyacrylamide gel. FIG. 7B shows a Western blot of the gel in panel A probed with anti-TcdB mAb 5A8E11. Samples in lanes 1-5 are nap_3 mTcdB expressed in spoOA-A, lane 6 is nap_3 mTcdB expressed in WH320, lane 7 is native TcdB (List Biologics) and lane 8 is Hi-Mark Molecular Weight Marker (Invitrogen).

FIG. 8 shows a schematic of the xylose utilization operon (xylABT operon, 5351 bp), plus xylR, which is orientated in the opposite direction as xylABT. Shown are the locations of the genes for xylose repressor (xylR), xylose isomerase (xylA), xylulokinase (xylB) and xylose transporter (xylT). The xylR promoter overlaps that of xylABT.

FIG. 9 provides the nucleotide sequence of the erythromycin resistance marker ermC under the control of P-dfrA (SEQ ID NO:3). The P-drfA nucleotide sequence is underlined. A BamHI restriction site, used to add the resistance marker to the construct, is shown in bold, italics.

FIG. 10 provides the nucleotide sequence of the xylAR knockout cassette (SEQ ID NO:4). The sequence of P-drfA-ermC is underlined. KpnI and XmaI restriction sites are shown in bold, italics.

FIG. 11 shows the possible integration events resulting from the integration protocol used to knock-out the xylR and xylA genes from the ΔspoOA-A knock-out B. megaterium strain, as described in Examples 5-7. Pictured in FIG. 11A is chromosomal DNA from B. megaterium ΔspoOA-A strain with a region 3′ of xylR genes and xylB gene (3′ flanking relative to xylA) indicated. Below the chromosomal DNA is the pCL10 integration vector, in which the ermC resistance marker flanked by the upstream and downstream regions of xylR/xylA (3′ xylR flanking sequence and xylB sequence) have been inserted. FIG. 11B shows the ΔspoOA-A B. megaterium chromosomal DNA following a single cross-over event, which leads to integration of the entire plasmid and maintenance of erythromycin and chloramphenicol resistance. Functional copies of xylR and xylA are also maintained. FIG. 11C shows B. megaterium chromosomal DNA following a double cross-over event, which leads to the integration of only the ermC marker. In the case of a double cross-over, erythromycin resistance is maintained and chloramphenicol resistance is lost. In this case xylA and xylR are removed from the chromosomal DNA.

FIG. 12 shows expression of nap_5 mTcdB in a ΔspoOΔAxylAR strain. FIG. 12A shows samples run on a polyacrylamide gel. FIG. 12B shows a Western blot of the gel in FIG. 12A probed with anti-TcdB mAb 5A8E11. Samples in lanes 1-8 are nap_5 mTcdB expressed in ΔspoOΔAxylAR1, lane 9 is native TcdB (List Biologics) and lane 10 is Hi-Mark Molecular Weight Marker (Invitrogen)

FIG. 13 shows a schematic of the B. megaterium Ilv-leu operon (8209 bp), which is composed of seven genes that are responsible for the biosynthesis of the branched amino acids: leucine, isoleucine and valine.

FIG. 14 provides the nucleotide sequence of the leuCD knockout cassette (SEQ ID NO:5). The sequence of leuB is underlined and a BamHI restriction site is shown in bold, italics. In this example, a resistance marker was not inserted between the 5′ and 3′ flanking regions. Isolates which had undergone a double cross-over were unable to synthsize leucine. Isolates were streaked on M9 minimal media plates. Isolates which had undergone a double cross-over and were therefore leucine auxotrophs were unable to grown on minimal media. Isolates which maintained the plasmid or had undergone a single cross-over event were able to biosynthesize leucine and thus remained viable when grown on minimal media. Four isolates were leucine auxotrophs (B.megΔleuCD A-D). These isolates also maintained the characteristics of the previous mutational work performed in the parent strains and thus were sporulation deficient and unable to metabolize xylose.

FIG. 15 shows the possible integration events resulting from the integration protocol used to knock-out the leuC and leuD genes from the ΔspoOΔAxylAR1 B. megaterium strain chromosomal DNA, as described in Example 9. Pictured in FIG. 15A is chromosomal DNA from B. megaterium ΔspoOΔAxylAR1strain with the leuCD, leuB and the 3′ flanking region indicated. Below the chromosomal DNA is the pCL10 integration vector with a knockout cassette consisting of a fragment at the 3′ region of leuB and a fragment at the region downstream from leuD. FIG. 11B shows the ΔspoOΔAxylAR1 DNA following a single cross-over event, which leads to integration of the entire plasmid, and maintenance of chloramphenicol resistance. In the case of a single cross-over, isolates remain leucine autotrophs. FIG. 11C shows ΔspoOΔAxylAR1 DNA following a double cross-over event, which leads to the deletion of leuCD genes and loss of chloramphenicol resistance.

FIG. 16 provides the nucleotide sequence of the predicted ilv-leu promoter, Pleu, in B. megaterium, which was predicted based on alignment of previously characterized ilv-leu promoter of B. subtilis (see Example 10, SEQ ID NO:6). Underlined nucleotides indicate the predicted −10 and −35 regions.

FIG. 17 shows the predicted structure of stem1 in the likely region of the ilv-leu promoter of B. megaterium. The location of stem 1 was predicted based on RNAfold analysis of secondary structure for a region homologous to the location of stem1 previously described for the B. subtilis promoter.

FIG. 18 shows the predicted structure of the terminator in the likely region of the ilv-leu promoter of B. megaterium (see Example 10).

FIG. 19 provides the nucleotide sequences of mutant promoters P-503 (panel A, SEQ ID NO:7), P-201 (panel B, SEQ ID NO:8), and P-306 (panel C, SEQ ID NO:9).

FIG. 20 provides the nucleotide sequence of wild-type leuCD plus additional nucleotides comprising a predicted transcriptional terminator, which are underlined (SEQ ID NO:10).

FIG. 21 provides the nucleotide sequence of the P201leu construct (P201+leuCD+terminator; SEQ ID NO:11). The sequence of P201 is underlined. The predicted transcriptional terminator is double-underlined.

FIG. 22 provides the nucleotide sequence of the Pleuleu construct (P-leu (wt)+leuCD+terminator; SEQ ID NO:12). The nucleotide sequence of P-leu is underlined. The predicted transcriptional terminator is double-underlined.

FIG. 23 provides the nucleotide sequence of the P503leu construct (P-503+leuCD+terminator; SEQ ID NO:13). The P-503 sequence is underlined. The predicted transcriptional terminator is double-underlined.

FIG. 24 provides the nucleotide sequence of the P306leu construct (P-306+leuCD+terminator; SEQ ID NO:14). The sequence of P-306 is underlined. The predicted transcriptional terminator is double-underlined.

FIG. 25 provides an example of a 3mCDTa expression plasmid containing a leuCD cassette (see Example 11). Panel A shows P306-3mCDTa2-pMM1522, which contain variant promoter P306 and leuCD and panel B shows the parent plasmid pMM1522. Expression was analyzed by Western blot and Coomassie stained gel.

FIG. 26 shows expression in a leucine auxotroph of CDTa after 20 hour induction. Shown in panel A are samples run on a polyacrylamide gel and stained with Coomassie Blue. Panel B shows a Western blot of the gel in panel A probed with pooled anti-CDTa hamster serum. Hamsters were immunized 4 times with CDTa formulated with Isomatrix. Two weeks after the final immunization hamsters were bleed. Serum samples were pooled and used as a primary antibody for the Western blots. Samples were grown in either M9 modified media or LB media. M9 modified media contained 1×M9 salts (Invitrogen), 20 g/L glycerol, 1 mM MgSO₄ (Sigma-Aldrich), 0.1 mM CaCl₂ (Sigma-Aldrich), and trace elements. Samples in lanes 1-4 were grown in M9 modified media and samples in lanes 4-8 were grown in LB media. Samples are as follows: lane 1-P306leuCD-3mCDTa2-pMM1522; lane 2-Pleu-1leuCD-3mCDTa2-pMM1522; lane 3-P201leuCD-3mCDTa2-pMM1522; lane 4-B.meg ΔleuCD D control; lane 5-P306leuCD-3mCDTa2-pMM1522; lane 6-Pleu-1leuCD-3mCDTa2-pMM1522; lane 7-P201leuCD-3mCDTa2-pMM1522; lane 8-B.megΔleuCD control; and lane 9-molecular weight marker.

FIG. 27 shows expression of a leucine auxotroph CDTa after a 5-hour induction. Shown in panel A are samples run on a polyacrylamide gel and stained with Coomassie Blue. Panel B shows a Western blot of the gel in panel A probed with pooled anti-CDTa hamster serum, as described above. Samples are the same as in FIG. 26.

FIG. 28 shows a schematic of pleuleu expression vectors without tetR.

FIG. 29 shows a schematic of pleuleu expression vectors with tetR.

### DETAILED DESCRIPTION OF THE INVENTION

The invention relates to a B. megaterium expression system that comprises a modified B. megaterium strain and an expression vector, wherein the expression system comprises one or more of the following modifications relative to known B. megaterium expression systems: (1) sporulation is knocked out by a deletion or functional deletion of spo0A in the B. megaterium genome; (2) xylose utilization (i.e. ability to metabolize xylose) is knocked out by a deletion or functional deletion of the genes xylA and xylR in the B. megaterium genome; and (3) the system utilizes leucine biosynthesis complementation. In a preferred B. megaterium expression system of the invention, leucine biosynthesis complementation is achieved by engineering a leucine auxotrophic B. megaterium strain, e.g. by knocking out one or more genes from the Ilv-leu operon such as the leuC and leuD genes from the B. megaterium genome and conversely adding the same genes (e.g. leuC and leuD) to an expression plasmid introduced into the B. megaterium strain.

There are several advantages associated with use of B. megaterium for recombinant expression of heterologous proteins. Benefits include expression of only one secreted protease and no alkaline proteases, or endotoxin by B. megaterium. Additionally, plasmids can be stably maintained in a B. megaterium expression system. In one study, B. megaterium strain DSM319, which does not contain endogenous plasmids, was shown to maintain introduced plasmids for 3 weeks in 80-100% of cells without selective pressure (Meinhardt et al. (1989), supra). Strain QM B1551 has been shown to maintain endogenous plasmids for over 100 generations with no selection or loss (Vary et al., Bacillus megaterium-from simple soil bacterium to industrial protein production host. Appl. Microbiol. Biotechnol. 76: 957-967 (2007)). Strain QM B1551 harbors seven plasmids and has been reported to stably carry over 11% of its cellular DNA in plasmids (Kieselburg et al., Analysis of resident and transformant plasmids in Bacillus megaterium. Bio/Technology 2:254-259 (1984)). Most strains of B. megaterium carry multiple plasmids, some greater than 100 kb.

Despite the advantages of B. megaterium noted above, known B. megaterium expression systems are not optimal for use as manufacturing platforms for recombinant protein production because of the requirement for the use of antibiotics for selection and maintenance of the expression plasmids and the difficulties associated with working with a sporulating bacterium as a cell substrate. Sporulation is a survival mechanism induced in certain types of bacteria when the bacterium encounters adverse conditions unfavorable for its survival, such as starvation. Under such adverse conditions, these baceteria disperse spores, which are very difficult to eradicate. Because of the contamination risk associated with sporulating bacteria, it is not desirable to use them for production on an industrial scale. Thus, the invention provides B. megaterium expression systems which do not form spores and thus are an improvement over known expression systems and methods for expressing heterologous nucleotide sequences using said expression systems.

Bacillus megaterium Strains

The invention relates to an improved Bacillus megaterium expression system that comprises a B. megaterium strain (i.e. population of cells) that is modified to functionally eliminate sporulation as discussed, infra. The asporogenous strains of the invention are derived from a B. megaterium progenitor strain, which has been modified to delete or functionally delete the SpoOA gene. The progenitor strain can be any B. megaterium wild-type strain, which is then modified to produce an asporogenous strain of the invention, or it can be an already modified B. megaterium strain that is further modified to delete or functionally delete SpoOA. In embodiments of the invention, the progenitor strain is DSM319. In alternative embodiments, the progenitor strain is a DSM319 mutant or derivative strain (i.e. a DSM 319 strain that has one or more genetic modifications such as one or more deletions, substitutions, or insertions, or combinations thereof, in its genome relative to wild-type DSM319), including, but not limited to, those strains listed in Table 2 (See Vary et al, 2007, supra). In certain embodiments, the DSM319 mutant strain that serves as the progenitor strain may comprise mutations or deletions of genes known to be involved in sporulation which are not SpoOA.

TABLE 2
Mutants generated in B. megaterium strain DSM319

| Strain | Description | Reference |
|---|---|---|
| spo3::Tn917 | spo3::Tn917 spo− | Meinhardt et al. (1994) Appl. Microbio.l Biotechnol. |
| | | 41: 244-351 |
| MS941 | ΔnprM | Appl. Microbiol. Biotechnol. 44: 317-326 |
| MS942* | ΔnprM, ΔleuC | Wittchen et al. (1998) J. Gen. Appl. Microbiol., |
| | | 44, p 317-326 |
| MS943* | ΔnprM, ΔleuC, ΔspoIV | Wittchen et al. (1998), supra |
| MS944* | ΔnprM, ΔleuC, ΔspoIV, ΔrecA | Wittchen and Meinhardt (1995), supra |
| MS961* | ΔleuC::nprM | Unpublished |
| MS970 | bgaR::bgl | Strey et al. (1999) J. Bacteriol. 181: 3288-3292 |
| MS971 | ΔbgaR | Strey et al. (1999), supra |
| MS972 | ORF2::bgl | Strey et al. (1999), supra |
| MS981 | bgaM::nprM | Strey et al. (1999), supra |
| MS982 | ΔbamM | Lee et al. (2001), Appl. Microbiol. Biotechnol. |
| | | 56: 205-211 |
| MS983 | ΔnprM, ΔleuC, ΔbgaR/bgaM | Unpublished |
| MS991 | ΔrecA1 | Nahrstedt et al. (2005) Microbiol. 151: 775-787 |
| MS001 | ΔnprM, ΔbarM/bamM | Unpublished |
| MS011 | ΔuvrA::bgl | Nahrstedt and Meinhardt (2004) Appl. Microbiol. |
| | | Biotechnol. 64: 243-249 |
| MS021 | ΔbgaR/bgaM | Schmidt et al. (2005) Appl. Microbiol. Biotechnol. |
| | | 68: 647-655 |
| MS022 | ΔuvrBA::cat | Nahrstedt and Meinhardt (2004), supra |
| MS023 | ΔbgaR/bgaM, leuC:: bgaM | Schmidt et al. (2005), supra |
| MS033 | ΔuvrB | Nahrstedt and Meinhardt (2004), supra |
| WH320 | LacZ | Rygus et al. (1991) Arch. Microbiol., 155(6): 535-42 |
| WH321 | Lac xylR Kn^{r} | Rygus and Hillen (1992) J. Bacteriol., 174(9): 3049-3055 |
| WH322 | Lac Kn^{r} xylR xylA1-spoVG- | Rygus and Hillen (1992), supra |
| | lacZ | |
| WH323 | XylA1-spoVG-lacZ | Rygus and Hillen (1992), supra |
| WH324 | XylA2-spoVG-lacZ | Rygus and Hillen (1992), supra |
| WH325 | Lac Kn^{r} xylR xylA2-spoVG- | Rygus and Hillen (1992), supra |
| | lacZ | |

*Due to a change in B. megaterium gene nomenclature over time, the “leuC” gene that was deleted from strains MS942, MS943, and MS944 is the 3-isopropylmalate dehydrogenase gene, rather than the large subunit of 3-isopropylmalate dehydratase, which was knocked-out in the Examples herein. It is unknown which gene was referred to as leuC in strain MS961.

In still other embodiments, the progenitor strain from which the asporogenous B. megaterium strains of the invention are derived is a QM B1551 strain. In further embodiments, the progenitor strain is a QM B1551 mutant or derivative strain (i.e., a QM B1551 strain that has a genetic modification such as a deletion, substitution, or insertion in its genome relative to wild-type QM B1551). QM B1551 mutant strains that are useful as progenitor strains of the invention, include, but are not limited to, mutant strains comprising Tn917 insertion mutations in sporulation genes spoIIA (strains PV517 and PV518) and spoIIAC, coding for sigma factor F, (strains PV519), and a lac negative mutant (strain PV586) (Tao et al. 1992; Tao and Vary 1991).

### Sporulation

Despite the advantages of using B. megaterium as a component of an expression system, spore production by native bacteria makes this system unsuitable for commercial protein production. Spo0A plays a critical role in the initiation of sporulation (Molle et al., The Spo0A regulon of Bacillus subtilis. Molecular Microbiology 50(5), 1683-1701 (2003)). Spo0A is activated via phosphorylation during the transition to sporulation. Once phosphorylated, it binds to a DNA sequence called the 0A box (also known as the spo0A box) where it acts to repress certain vegetative genes and activate other genes in the sporulation cascade. In Bacillus subtilis, it was shown that Spo0A directly regulates the transcription of 121 genes and influences the expression of over 500 genes (Fawcett et al., The transcriptional profile of early to middle sporulation in Bacillus subtilis. Proc. Natl. Acad. Sci. USA 97(4): 8063-8068 (2000); Molle et al. (2003), supra). Spo0A activates genes involved in remodeling the sister chromosomes of sporulating cells into an axial filament (Pogliano et al., Partitioning of chromosomal DNA during establishment of cellular asymmetry in Bacillus subtilis. J. Bacteriol. 184(6). 1743-9 (2002)) as well as genes involved in the formation of the asymmetric septa, which divides the cell into two compartments that eventually develop into the mother cell and the forespore (Levin and Grossman, Cell cycle and sporulation in Bacillus subtilis. Current Opinion in Microbiology 1: 630-635 (1998); Pogliano et al., 2002, supra). Spo0A null mutants in Clostridium perfringens have been shown to be unable to form endospores (Huang et al. FEMS Microbiology Letters 233: 233-240 (2004)). Under sporulation conditions, chromosomal segregation occurs as it did in vegetative growth and cells divide by binary fission (Dunn et al., J. Bacteriol. 123(3):776-779 (1976); Levin and Losick, Transcription factor Spo0A switches the localization of cell division protein FtsZ from medial to a bipolar pattern in Bacillus subtilis. Genes Dev. 10:478-488 (1996)).

To that end, the present invention relates to a Bacillus megaterium strain that is deficient in sporulation. In particular, the invention provides a B. megaterium strain that has a knock-out of the Spo0A gene (i.e. is deficient in or comprises a deletion or functional deletion of Spo0A). Thus, the invention is related to an isolated or purified asporogenous Bacillus megaterium (B. megaterium) genome, wherein the B. megaterium genome comprises a deletion or functional deletion of the spo0A gene (i.e. wherein the B. megaterium genome has been modified so that the spo0A gene is deleted or functionally deleted) and the strain does not produce spores. In particular embodiments, the SpoOA gene is deleted. In embodiments of the invention wherein DSM319 or a DSM319 derivative is the progenitor strain, the spo0A gene that is deleted or disrupted (i.e. functionally deleted) from the B. megaterium genome comprises a sequence of nucleotides as set forth in SEQ ID NO:54, which is the spo0A nucleotide sequence in the DSM319 strain. One of skill in the art will realize that the spoOA gene to be functionally deleted may comprise a sequence of nucleotides that is substantially similar to SEQ ID NO:54, i.e. a sequence that has 90%, 92%, 94%, 95%, 96%, 97%, 98% or 99% or greater homology with the nucleotide sequence set forth in SEQ ID NO:54. In alternative embodiments wherein the progenitor B. megaterium strain is from a different B. megaterium strain (i.e. not DSM319), the spo0A gene to be functionally deleted comprises a sequence of nucleotides that has 90%, 92%, 94%, 95%, 96%, 97%, 98% or 99% or greater homology to the nucleotide sequence set forth in SEQ ID NO:54.

Asporogenous strains of Bacillus subtilis have been described (U.S. Pat. Nos. 6,284,490; 4,302,544; 4,450,235; and 4,465,773). Meinhardt et al. (Cloning and sequencing of the leuC and nprM genes and a putative spoIV gene from Bacillus megaterium DSM319. Appl. Microbiol. Biotechnol. 41:344-351 (1994)) described a Tn917-derived sporulation deficient mutant of B. megaterium DSM319 (spo3::Tn917) in which they disrupted the putative spoIV gene. Wittchen et al. (Molecular characterization of the operon comprising the spoIV gene of Bacillus megaterium DSM319 and generation of a deletion mutant. J. Gen. Appl. Microbiol., 44: 317-326 (1998)) characterized the spoIV locus and generated the deletion strain MS943 (ΔnprM, ΔleuC, ΔspoIV). However, microscopic analysis of MS943 revealed that, due to the deletion of a late stage sporulation gene, nonfunctional, immature prespores were formed and released from mother cells. MS943 was described as phenotypically identical to spo3::Tn917 (Wittchen et al., 1998, supra). The present invention, on the other hand, utilizes a knock-out of spo0A rather than spoIV to eliminate sporulation because spoOA deletion does not lead to the formation of prespores, which could potentially block microscopic detection in the event a culture becomes contaminated with a sporulating B. megaterium strain.

### Xylose Utilization

The invention also relates to an asporogenous B. megaterium strain that comprises a B. megaterium genome, wherein said genome is modified by a deletion or functional deletion of the spoOA gene and wherein the genome is further modified by either: (1) a deletion or functional deletion of the xylA and xylR genes, (2) a deletion or functional deletion of one or more genes in the ilv-leu operon necessary to produce leucine, e.g. the leuC and leuD genes, or (3) the deletions or functional deletions described in (1) and (2), wherein the strain does not produce spores. In embodiments of the invention where xylA and xylR are deleted or functionally deleted, the modified B. megaterium strain of the invention cannot metabolize xylose, as described below. In embodiments of the invention where one or more genes from the Ilv-leu operon are deleted or functional deleted, the modified B. megaterium strain of the invention does not synthesize leucine. In embodiments of the invention wherein leuC and leuD are deleted or functionally deleted, the modified B. megaterium strain does not express the large and small subunits of 3-isopropylmalate dehydratase, thus allowing utilization of leucine complementation by incorporation of these genes into an expression vector to be used in conjunction with the modified cells in an expression system.

Bacillus megaterium can survive utilizing xylose as the sole carbon source. Known expression systems utilize B. megaterium substrates that metabolize xylose, which is used to induce expression, making it difficult to maintain a steady state during fermentations. It is shown herein that a B. megaterium strain in which the xylA and xylR genes are knocked-out cannot metabolize xylose. This lack of xylose metabolism is advantageous in a recombinant expression system because it allows control over expression of recombinant proteins from the cell by allowing a steady state to be reached during fermentation. To that end, the asporogenous B. megaterium strains of the invention may be further modified to knock-out xylose metabolism through a deletion or functional deletion of xylA and xylR. Thus, the invention is related to an asporogenous B. megaterium strain that comprises a deletion or functional deletion of spo0A, xylA and xylR, wherein the strain is unable to sporulate and unable to metabolize xylose.

Accordingly, the invention provides an isolated or purified asporogenous B. megaterium strain that comprises a genome that has a deletion or functional deletion of spo0A, as described in any embodiment herein, and a deletion or functional deletion of xylA and xylR. In particular embodiments, the xylA gene is deleted and the xylR gene is functionally deleted. In other embodiments, the xylA gene is functionally deleted and the xylR gene is deleted. In further embodiments, the xylA and the xylR genes are either both deleted or both functionally deleted. In specific embodiments wherein xylA and/or xylR genes are functionally deleted, the functional deletion is a deletion of one or more nucleotides from the gene(s) which renders the encoded gene product non-functional, i.e. deficient in xylose utilization. In other embodiments wherein xylA and/or xylR genes are functionally deleted, the functional deletion is an insertion of one or more nucleotides into the gene(s) which renders the encoded gene product non-functional. In still other embodiments, the xylA and/or xylR genes comprise one or more point mutations which render the gene product non-functional, for example, one or multiple non-conservative substitutions. In further embodiment of the invention, the complete gene sequence of xylA and/or xylR is deleted.

In embodiments of the invention wherein DSM319 or a DSM319 derivative is the progenitor strain, the xylA gene that is deleted or functionally deleted from the B. megaterium genome comprises a sequence of nucleotides as set forth in SEQ ID NO:55, which is the xylA nucleotide sequence in the DSM319 strain. One of skill in the art will realize that the xylA gene to be deleted or functionally deleted may comprise a sequence of nucleotides that is substantially similar to SEQ ID NO:55, i.e. a sequence that has 90%, 92%, 94%, 95%, 96%, 97%, 98% or 99% or greater homology to the nucleotide sequence set forth in SEQ ID NO:55. In alternative embodiments wherein the progenitor B. megaterium strain is from a different B. megaterium strain (i.e. not DSM319), the xylA gene to be functionally deleted comprises a sequence of nucleotides that has 90%, 92%, 94%, 95%, 96%, 97%, 98% or 99% or greater homology to the nucleotide sequence set forth in SEQ ID NO:55.

In embodiments of the invention wherein DSM319 or a DSM319 derivative is the progenitor strain, the xylR gene that is deleted or functionally deleted from the B. megaterium genome comprises a sequence of nucleotides as set forth in SEQ ID NO:56, which is the xylR nucleotide sequence in the DSM319 strain. One of skill in the art will realize that the xylR gene to be deleted or functionally deleted may comprise a sequence of nucleotides that is substantially similar to SEQ ID NO:56, i.e. a sequence that has 90%, 92%, 94%, 95%, 96%, 97%, 98% or 99% or greater homology to the nucleotide sequence set forth in SEQ ID NO:56. In alternative embodiments wherein the progenitor B. megaterium strain is from a different B. megaterium strain (i.e. not DSM319), the xylR gene to be functionally deleted comprises a sequence of nucleotides that has 90%, 92%, 94%, 95%, 96%, 97%, 98% or 99% or greater homology with the nucleotide sequence set forth in SEQ ID NO:56.

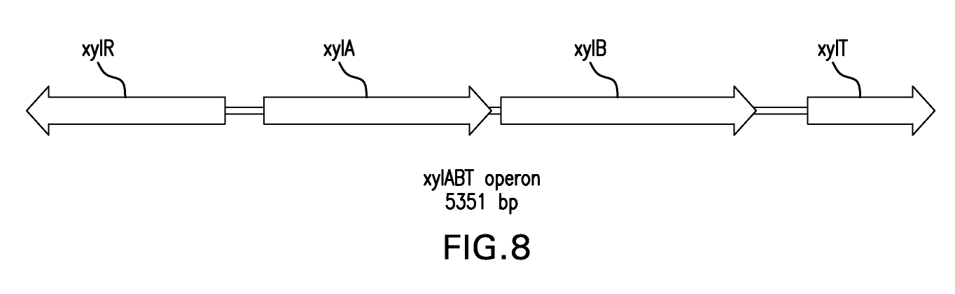

Rygus and colleagues (Arch. Microbiol., 155(6): 535-42 (1991)) initially characterized two genes of the xylose operon (see FIG. 8 for schematic of the xylose utilization operon (xylABT operon) and xylR) of Bacillus megaterium: xylA, encoding the xylose isomerase and xylB, encoding xylulokinase. Schmiedel et al. (Regulation of expression, genetic organization and substrate specificity of xylose uptake in Bacillus megaterium. Mol. Microbiol. 23(5): 1053-1062 (1997)) later described an additional gene in the operon: xylT, which expresses a putative H+/xylose symporter. Expression of the xylose operon was found to be tightly regulated by the xyl repressor (XylR) encoded by xylR. In the absence of xylose, XylR binds to the xylA promoter at two overlapping operator sequences preventing transcription of the operon. In the presence of xylose, xylose binds to XylR causing a conformational change and preventing it from binding to the promoter and allowing transcription of the xylABT operon (Schmiedel et al. (1997), supra, Rygus et al. (1991), supra). Schmiedel et al. (1997, supra) reported that Northern blot analysis revealed two different RNA transcripts containing xylA: one which ends with xylA and which is transcribed through xylT. It is thought that a palindromic sequence between xylA and xylB can function as a transcriptional terminator, which leads to the truncation of more than 50% of the RNA transcripts produced rather than their continuation through xylBT (Schmiedel et al., Regulation of expression, genetic organization and substrate specificity of xylose uptake in Bacillus megaterium. Mol. Microbiol. 23(5):1053-1062 (1997)).

Mutants of the xylose operon have been described. Rygus and Hillen (1992) generated deletion strains WH322, WH323, WH324 and WH325 (see Table 2) to study catabolite repression. Yang et al (BMC Microbiology, 8:192 (2006)) generated a knockout strain, YYBm1, deficient for xylose utilization by interrupting the xylA gene in strain MS941 (ΔnprM). Strains deficient for xylose utilization have also been used by Biedendieck et al., (Biotechnol Bioeng. 96(3): 525-537 (2007)) and Schmidt et al (Appl. Microbiol. Biotechnol. 68:647-655 (2005).

### Leucine Biosynthesis Complementation

The invention also relates to an asporogogenous B. megaterium strain that comprises a B. megaterium genome, wherein said genome is modified by a deletion or functional deletion of the spoOA gene and wherein the genome is further modified by a deletion or functional deletion of one or more genes from the Ilv-leu operon, wherein the strain is leucine auxotrophic. In particular embodiments, the leuC and leuD genes are deleted or functionally deleted. In particular embodiments of the invention the leuC and leuD are deleted or functionally deleted, and the modified B. megaterium strain of the invention cannot synthesize leucine (i.e. the strain is leucine auxotrophic), as described below. In embodiments of the invention where leuC and leuD are deleted or functionally deleted, the modified B. megaterium strain of the invention does not express functional large and small subunits of 3-isopropylmalate dehydratase, thus allowing utilization of leucine complementation by incorporation of these genes into an expression vector to be used in conjunction with the modified cells in an expression system.

Accordingly, the invention provides an isolated or purified asporogenous B. megaterium strain that comprises a genome that has a deletion or functional deletion of spo0A, as described in any embodiment herein, and/or a deletion or functional deletion of one or more genes from the Ilv-leu operon selected from the group consisting of: leuD, leuC, leuB, and leuA; wherein the strain is leucine auxotrophic. In preferred embodiments of the invention, the leuC and/or leuD genes are deleted or functionally deleted from the genome of the modified B. megaterium strain. Knocking out or inactivating either or both of the leuC and leuD genes blocks key steps in the leucine biosynthesis pathway. In alternative embodiments, the leuB gene is deleted or functionally deleted. In further embodiments, the leuA gene is deleted or functionally deleted. In other embodiments, the B. megaterium genome comprises a deletion or functional deletion in the leuB and/or leuA genes in addition to a deletion or functional deletion of the leuC and leuD genes.

In particular embodiments of the invention, the leuC and/or leuD genes are deleted or functionally deleted. In particular embodiments, the leuC gene is deleted and the leuD gene is functionally deleted. In other embodiments, the leuC gene is functionally deleted and the leuD gene is deleted. In further embodiments, the leuC and the leuD genes are either both deleted or both functionally deleted. In specific embodiments wherein leuC and/or leuD genes are functionally deleted, the functional deletion is a deletion of one or more nucleotides from the gene(s) which renders the encoded gene product non-functional, i.e. the functional deletion renders the bacteria unable to perform leucine biosynthesis. In other embodiments wherein leuC and/or leuD genes are functionally deleted, the functional deletion is an insertion of one or more nucleotides into the gene(s) which renders the encoded gene product non-functional. In still other embodiments, the leuC and/or leuD genes comprise one or more point mutations which render the gene product non-functional, for example, one or multiple non-conservative substitutions. In further embodiment of the invention, the complete gene sequence of leuC and/or leuD is deleted.

In embodiments of the invention wherein DSM319 or a DSM319 derivative is the progenitor strain, the leuC gene that is deleted or functionally deleted from the B. megaterium genome comprises a sequence of nucleotides as set forth in SEQ ID NO:57, which is the leuC nucleotide sequence in the DSM319 strain. One of skill in the art will realize that the leuC gene to be deleted or functionally deleted may comprise a sequence of nucleotides that is substantially similar to SEQ ID NO:57, i.e. a sequence that has 90%, 92%, 94%, 95%, 96%, 97%, 98% or 99% or greater homology with the nucleotide sequence set forth in SEQ ID NO:57. In alternative embodiments wherein the progenitor B. megaterium strain is from a different B. megaterium strain (i.e. not DSM319), the leuC gene to be functionally deleted comprises a sequence of nucleotides that has 90%, 92%, 94%, 95%, 96%, 97%, 98% or 99% or greater homology with the nucleotide sequence set forth in SEQ ID NO:57.

In embodiments of the invention wherein DSM319 or a DSM319 derivative is the progenitor strain, the leuD gene that is deleted or functionally deleted from the B. megaterium genome comprises a sequence of nucleotides as set forth in SEQ ID NO:58, which is the leuD nucleotide sequence in the DSM319 strain. One of skill in the art will realize that the leuD gene to be deleted or functionally deleted may comprise a sequence of nucleotides that is substantially similar to SEQ ID NO:58, i.e. a sequence that has 90%, 92%, 94%, 95%, 96%, 97%, 98% or 99% or greater homology with the nucleotide sequence set forth in SEQ ID NO:58. In alternative embodiments wherein the progenitor B. megaterium strain is from a different B. megaterium strain (i.e. not DSM319), the leuD gene to be functionally deleted comprises a sequence of nucleotides that has 90%, 92%, 94%, 95%, 96%, 97%, 98% or 99% or greater homology to the nucleotide sequence set forth in SEQ ID NO:58.

### Expression Vectors

In another aspect, the invention provides a B. megaterium cell as described in any embodiment herein, further comprising an expression vector comprising a sequence of nucleotides in the form of a transgene that encodes a protein of interest (i.e. an expression system). The nucleic acid sequence embodying the transgene can be a gene, or a functional part of a gene and will typically exist in the form of an expression cassette. Typically a gene expression cassette includes: (a) nucleic acid encoding a protein of interest; (b) a heterologous promoter operatively linked to the nucleic acid encoding the protein; and (c) a transcription termination signal. The nucleic acid can be codon-optimized for expression in the desired host (e.g., B. megaterium). Generally speaking, the transgene which is cloned into the expression vector and transformed into the B. megaterium cells of the invention encodes a polypeptide, protein, or enzyme product of interest. Said transgene comprises a nucleotide sequence which provides the necessary regulatory sequences to direct transcription and/or translation of the encoded product in a B. megaterium host cell of the invention.

The protein or antigen of interest may be any protein or protein fragment for which recombinant expression is desired. The expression system can be evaluated in vitro for the levels of heterologous polypeptide expression using techniques known in the art (including, but not limited to, enzyme-linked immune assay, immunocytochemistry, immunoblots, FACS, etc.).

In embodiments of the invention, the heterologous polypeptide/protein of interest is an immunogen (antigenic molecule), which comprises a polypeptide, protein, or enzyme product that is encoded by a transgene (i.e. heterologous nucleotide sequence) in combination with a nucleotide sequence which provides the necessary regulatory sequences to direct transcription and/or translation of the encoded product in a modified host cell of the invention. In an embodiment of the invention, the heterologous polypeptide expressed by the expression system is a polypeptide that is useful in the treatment and/or prevention of a pathology. In one embodiment, the pathology is one that can be prevented and/or treated by an immunologic response. In specific embodiments, the heterologous polypeptide is an antigen useful in eliciting an immunological response (e.g., eliciting antibodies, CD4⁺ T cells and/or CD 8⁺ T cells) for preventing and/or treating a disorder (i.e. viral, bacterial or parasitic infection, or oncologic disorder). In other specific embodiments, the heterologous polypeptide is an antibody or portion thereof useful in passive immunity. In another embodiment, the heterologous polypeptide is a polypeptide useful in gene therapy.

The expression vector may also comprise appropriate transcriptional regulatory elements that are capable of directing expression of the transgene in the B. megaterium host cells that the vector is being prepared for use. Such regulatory elements are operatively linked to the transgene. “Operatively linked” sequences include both expression control sequences that are contiguous with the nucleic acid sequences that they regulate and regulatory sequences that act in trans, or at a distance to control the regulated nucleic acid sequence.

Regulatory sequences include: appropriate expression control sequences, such as transcription initiation, termination, enhancer and promoter sequences; efficient RNA processing signals, such as splicing and polyadenylation signals; sequences that enhance translation efficiency (e.g., Kozak consensus sequences); sequences that enhance protein stability, and optionally sequences that promote protein secretion. Selection of these and other common vector elements are conventional and many suitable sequences are well known to those of skill in the art (see, e.g., Sambrook et al, and references cited therein at, for example, pages 3.18-3.26 and 16.17-16.27 and Ausubel et al., Current Protocols in Molecular Biology, John Wiley & Sons, New York, 1989).

In specific embodiments, the promoter that is operatively linked to the nucleotide sequence encoding the protein of interest is a heterologous promoter. In some embodiments, the promoter is a “strong” or “efficient” promoter. An example of a strong promoter is the immediate early human cytomegalovirus promoter (Chapman et al, 1991 Nucl. Acids Res 19:3979-3986). The human CMV promoter can be used without (CMV) or with the intron A sequence (CMV-intA), although those skilled in the art will recognize that any of a number of other known promoters, such as the strong immunoglobulin, or other eukaryotic gene promoters may be used. Specific examples of promoters that can be used in the present invention are the EF1 alpha promoter, the murine CMV promoter, the Rous Sarcoma Virus promoter, the SV40 early/late promoters and the beta actin promoter, albeit those of skill in the art can appreciate that any promoter capable of effecting expression in the intended host can be used in accordance with the methods of the present invention. The promoter may comprise a regulatable sequence such as the Tet operator sequence. Sequences such as these that offer the potential for regulation of transcription and expression are useful in instances where repression of gene transcription is sought. Suitable gene expression cassettes will also comprise a transcription termination sequence. An exemplary transcriptional terminator is the bovine growth hormone terminator.

Also provided by the invention are modified expression vectors which are useful in conjunction with the modified B. megaterium strains of the invention or other bacterial host cells to form an expression system for the expression of heterologous proteins. In specific embodiments of the invention, the expression vector comprises the leuC and leuD genes, which allows efficient leucine complementation when transformed into a suitable B. megaterium strain of the invention, wherein the leuC and leuD genes have been deleted or functionally deleted. The leuC and leuD genes can be added to the vector using standard techniques well known in the art.

In alternative embodiments, the expression vector comprises the leuA and/or leuB genes, which allows efficient leucine complementation when transformed into a suitable B. megaterium strain of the invention, wherein the leuA and/or leuB genes have been deleted or functionally deleted. The leuA and leuB genes can be added to the vector using standard techniques well known in the art

In specific embodiments of the invention, the expression vector comprises a leuC gene that comprises a sequence of nucleotides as set forth in SEQ ID NO:57. In additional embodiments, the expression vector further comprises a leuD gene that comprises a sequence of nucleotides as set forth in SEQ ID NO:58. In further embodiments, the expression vector comprises a variant leuC gene that comprises a sequence of nucleotides that is at least 85%, at least 90%, at least 91%, at least 92%, at least 93%, at least 94%, at least 95%, at least 96%, at least 97%, at least 98%, or at least 99% or more homologous to the leuC sequence set forth in SEQ ID NO:57. In additional embodiments, the expression vector comprises a sequence of nucleotides that encodes the leuC polypeptide sequence set forth in SEQ ID NO:60, or encodes a variant of leuC that is at least 85%, at least 90%, at least 91%, at least 92%, at least 93%, at least 94%, at least 95%, at least 96%, at least 97%, at least 98%, or at least 99% or more identical to the amino acid sequence set forth in SEQ ID NO:60.

In still further embodiments, the expression vector comprises a variant leuD gene that comprises a sequence of nucleotides that is at least 85%, at least 90%, at least 91%, at least 92%, at least 93%, at least 94%, at least 95%, at least 96%, at least 97%, at least 98%, or at least 99% or more homologous to the leuD sequence set forth in SEQ ID NO:58. In additional embodiments, the expression vector comprises a sequence of nucleotides that encodes the leuD polypeptide sequence set forth in SEQ ID NO:61, or encodes a variant of leuD that is at least 85%, at least 90%, at least 91%, at least 92%, at least 93%, at least 94%, at least 95%, at least 96%, at least 97%, at least 98%, or at least 99% or more identical to the amino acid sequence set forth in SEQ ID NO:61. In some embodiments, the leuC and/or leuD sequences that are added to the expression vector comprise a variant of the sequence of nucleotides set forth in SEQ ID NO:57 or SEQ ID NO:58, wherein the variant nucleotide sequence encodes a polypeptide with one or more conservative amino acid substitutions relative to the polypeptide encoded by SEQ ID NO:57 or SEQ ID NO:58 (i.e. SEQ ID NO:60 and SEQ ID NO:61, respectively).

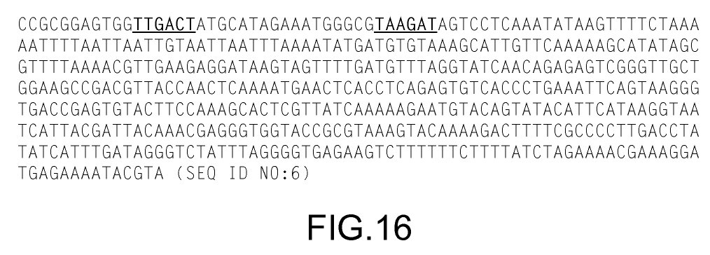

In some embodiments of the invention, the leuC and leuD nucleotide sequences are operatively linked to an Ilv-leu promoter or variant thereof. In specific embodiments of the invention, the ilv-leu promoter consists of, consists essentially of, or comprises a sequence of nucleotides as set forth in SEQ ID NO:6 (see also FIG. 16). In further embodiments, the leuC and leuD genes are operatively linked to a variant Ilv-leu promoter such as the P503 promoter, the P201 promoter, the P306 promoter, or the Pleu-1 promoter, which consist of, consist essentially of, or comprise a sequence of nucleotides as set forth in SEQ ID NO:7, SEQ ID NO:8, SEQ ID NO:9, or SEQ ID NO:59, respectively. In further embodiments, the promoter consists, consists essentially of, or comprises a sequence of nucleotides set forth in SEQ ID NO:7, SEQ ID NO:8, or SEQ ID NO:9. Three of the mutant promoters include deletions of the probable stem loop structures in each of the promoters. P201 (SEQ ID NO:8) contains a 233 base pair deletion in the region between the predicted site +26 of the transcription start site and the predicted protector is deleted from the leader sequence. P306 (SEQ ID NO:9) contains a 30 base pair deletion in the region between the predicted stem1 and protector. P503 (SEQ ID NO:7) contains a 153 base pair deletion from approximate 20 base pair upstream from the end of stem1 to the terminator. A fourth mutant promoter, Pleu-1 (SEQ ID NO:59) contains a single point mutation at nucleotide 15 relative to the wt leu promoter nucleotide sequence set forth in SEQ ID NO:6, in the predicted −35 sequence of the promoter.

In alternate embodiments, the leuC and/or leuD nucleotide sequences are operatively linked to an alternate variant Ilv-leu promoter, said alternate variant promoter comprising a sequence of nucleotides that is at least 85%, at least 90%, at least 91%, at least 92%, at least 93%, at least 94%, at least 95%, at least 96%, at least 97%, at least 98%, or at least 99% or more identical to the Ilv-leu promoter sequence set forth in SEQ ID NO:6 or the variant Ilv-leu promoter sequences set forth in SEQ ID NO:7, SEQ ID NO:8, SEQ ID NO:9 or SEQ ID NO:59.

In some embodiments of the invention, leuC and leuD are operatively linked to the same promoter. In other embodiments, leuC and leuD are operatively linked to two separate promoters.

In further embodiments of the invention, a transcriptional terminator is incorporated into the nucleotide sequence of the vector following nucleotide sequences encoding leuC and leuD. In some embodiments, the leuCD sequence+the transcriptional terminator comprises a sequence of nucleotides as set forth in SEQ ID NO:10 or comprises a nucleotide sequence that is at least 85%, at least 90%, at least 91%, at least 92%, at least 93%, at least 94%, at least 95%, at least 96%, at least 97%, at least 98%, or at least 99% or more identical to the nucleotide sequence set forth in SEQ ID NO:10.

The invention also provides constructs which comprise: (1) one or two ilv-leu promoters, (2) one or more sequences of nucleotides encoding leuC and leuD or variant leuC and/or leuD sequences, such as a leuC or a leuD nucleotide sequence that encodes a polypeptide with one or more conservative amino acid substitutions relative to the polypeptide encoded by a wild type leuC or leuD reference sequence, and (3) a transcriptional terminator sequence. In specific embodiments, the construct comprises a sequence of nucleotides as set forth in SEQ ID NO:11 (p201 promoter+wt leuCD+terminator), SEQ ID NO:12 (pleu promoter+wt leuCD+terminator), SEQ ID NO:13 (p503 promoter+wt leuCD+terminator), or SEQ ID NO:14 (p306 promoter+wt leuCD+terminator).

Any expression vector that can be transformed into the B. megaterium strains of the invention can be used as a base vector for introduction of the modifications noted above. Advantageously, several expression vectors that have been used successfully in combination with B. megaterium host cells have been described and are useful in the expression systems of the invention.

For example, Rygus and Hillen (Appl. Microbial. Biotechnol., 35: 594-599 (1991a); Appl. Microbiol. Biotechnol. 35: 594-599 (1991b)) described a system for high-level recombinant expression in Bacillus megaterium utilizing elements from the B. megaterium xylose utilization operon. They constructed a shuttle vector, pWH1520, containing the origin of replication and ampicillin resistance gene from pBR322 and the origin of replication, the gene repU and tetracycline resistance gene from the Bacillus cereus plasmid pBC16. Expression is controlled by the inducible xylA promoter and is tightly regulated by the xylose repressor (xylR). The expression plasmid was shown to increase production of four genes, gdhA from B. megaterium, lacZ from E. coli, mro from Acinetobacter calcoaceticus and human puk, from 130-350-fold. Expression was found to be tightly controlled and proteins stably expressed without proteolytic degradation. Thus, in some embodiments of the invention, the pWH1520 vector is modified in accordance with the invention (i.e. to add the leuA, leuB, or leuC and leuD genes or variants or combinations thereof) and used as an expression vector in a complete expression system comprising the modified B. megaterium strains of the invention.

In other embodiments of the invention, the expression vector is a variant of the pWH1520 vector, such as the pWH1520 variant (referred to as pMM1520) described by Malten et al. (Biotechnol Bioeng. 89(2): 206-218 (2005)). The pMM1520 variant expression vector comprises a multiple cloning site containing 15 unique restriction sites relative to pWH1520 and eliminates a cre mediating glucose-dependent catabolite repressor of Pxyl. It was shown to express dextransucrase DsrS when transformed into B. megaterium strain MS941 (mutant of DSM319, ΔnprM).

In still other embodiments, the base vector is a modified pMM1520 vector, such as pMM1522, pSTOP1522, pMM1525, pMM1533, pSTREP1522, pHIS1522, pSTREP1525, pHIS1525 or pSTREPHIS1525 (see Malten et al. Appl. Environ. Microbiol. 72(2): 1677-1679 (2006)), which are described below:
1. pMM1522 was constructed from pMM1520 using site directed mutagenesis to insert a BsrGI restriction site at the 5′ end of the multiple cloning site upstream of the xylA start codon.
2. pSTOP1522 was derived from pMM1522 and includes a stop codon directly downstream of the NaeI restriction site followed by additional NruI and AgeI sites.
3. pMM1525 was derived from pMM1522 and contains the B. megaterium extracellular LipA signal peptide (SPlipA) between the BsrGI and BstBI sites for secretion of the protein of interest.
4. pMM1533: pMM1525 with an additional BstBI site downstream of SPlipA.
5. pSTREP1522: pSTOP1522 derivative—vector for the intracellular production of StrepII-tagged proteins.
6. pHIS1522: pSTOP1522 derivative vector for intracellular production of His6-tagged proteins.
7. pSTREP1525: Vector for the secretion of recombinant Strep-tagged proteins.
8. pHIS1525: Vector for the secretion of recombinant His6-tagged proteins.
9. pSTREPHIS1525: Vector for the secretion of recombinant Strep-His6-tagged proteins.

In further embodiments, the base vector is further modified from the vectors above, such as the 1622 series of vectors (pSTOP1622, pC-HIS1622, pC-STREP1622, pN-STREP-Xa1622, pN-HIS-TEV1622, or pN-STREP-TEV1622), which vectors comprise a deletion of an 855 base pair AflII fragment from the vectors above, resulting in the elimination of a partial and inactive tetracycline resistance gene (Biedendiech et al. Biotechnol Bioeng. 92(3): 525-537 (2007)). The 1622 vectors are described below:
1. pSTOP1622: Same as pSTOP1522, but lacking 855 bp AflII restriction fragment.
2. pC-HIS1622: Same as pHIS1522, but lacking 855 bp AflII restriction fragment.
3. pC-STREP1622: A pSTOP1622 derivative vector for intracellular production of C-terminal Strep-tagged proteins in B. megaterium.
4. pN-STREP-Xa1622: Same as pSTREP1522, but lacking 855 bp AflII restriction fragment.
5. pN-HIS-TEV1622: A pSTOP1622 derivative vector for intracellular production of N-terminal His6-tagged proteins in B. megaterium.
6. pN-STREP-TEV1622: A pSTOP1622 derivative vector for intracellular production of N-terminal Strep-tagged proteins in B. megaterium.
   The pN-STREP-Xa1622, pN-HIS-TEV1622, and pN-STREP-TEV1622 vectors also contain proteolytic cleavage sites (Factor Xa or TEV) that can be used to remove the fusion tag.

In still further embodiments, the base vector comprises a modified xylA promoter, such as the p3STOP1622, p3STOP1623, or p3STOP1624 vectors described by Stammen et al. (Appl. Environ. Microbiol. 76, p. 4037-4046 (2010)), which lead to an increased level of recombinant protein expression in B. megaterium. These vectors contain modifications to the −10 and -35 sequences, which increase transcriptional initiation frequency, an added hairpin in the 5′ untranslated region which increases the half-life of the mRNA and an optimized ribosomal binding site, which increases the affinity of the ribosome to the mRNA. The resulting expression vectors are listed below:
1. p3STOP1622: pSTOP1622 derivative encoding three stop codons for all possible reading frames downstream of mcs.
2. p3STOP1623: p3STOP1622 derivative containing an additional PacI restriction site.
3. p3STOP1624: p3STOP1623 derivative containing an additional NheI restriction site.

One of skill in the art will be able to select the appropriate expression vector as a base vector for incorporation of the modifications noted above (e.g. addition of leuA, leuB, leuC and/or leuD) depending on the particular protein to be expressed and the desired level of expression. In preferred embodiments of the invention, the base vector is pMM1522, pHIS1522 or pSTOP1522.

Any of the vectors described herein can further comprise a tetracycline resistance gene (tet). Exemplary vectors of the invention that comprise a sequence of nucleotides that encodes leuCD, operatively linked to a modified ilv-leu promoter, and further comprise a tet gene include vectors that consist of, consist essentially of, or comprise a sequence of nucleotides as set forth in SEQ ID NO:45 (P306+leuCD, pMM1522+tet), SEQ ID NO:46 (P503+leuCD, pMM1522+tet), SEQ ID NO:47 (P306+leuCD, HIS1522+tet), SEQ ID NO:48 (P503 promoter+leuCD, HIS1522+tet), or SEQ ID NO:49 (P503+leuCD, p3STOP1623+tet). In further embodiments, the vector comprises a sequence of nucleotides that is at least 85%, at least 90%, at least 91%, at least 92%, at least 93%, at least 94%, at least 95%, at least 96%, at least 97%, at least 98%, or at least 99% or more identical to the nucleotide sequence set forth in SEQ ID NO:45, SEQ ID NO:46, SEQ ID NO:47, SEQ ID NO:48, or SEQ ID NO:49.

In alternative embodiments, the vector does not comprise a tetracycline resistance gene. Exemplary vectors of the invention that do not comprise a tet gene include vectors that consist of, consist essentially of, or comprise a sequence of nucleotides as set forth in SEQ ID NO:50 (P503+leuCD, pMM1522-tet), SEQ ID NO:51 (P306+leuCD, pMM1522-tet), SEQ ID NO:52 (P503+leuCD, pHIS1522-tet), or SEQ ID NO:53 (P306+leuCD, pHIS1522-tet). In further embodiments, the vector comprises a sequence of nucleotides that is at least 85%, at least 90%, at least 91%, at least 92%, at least 93%, at least 94%, at least 95%, at least 96%, at least 97%, at least 98%, or at least 99% or more identical to the nucleotide sequence set forth in SEQ ID NO:50, SEQ ID NO:51, SEQ ID NO:52, or SEQ ID NO:53.

Homologous and Heterologous Expression in B. megaterium

In another aspect, the invention relates to a method of producing a recombinant, heterologous protein in a B. megaterium host cell comprising: (a) transforming a modified B. megaterium cell with an expression vector comprising a sequence of nucleotides that encodes the heterologous protein, wherein the modified cell comprises a B. megaterium genome that comprises a deletion or functional deletion of the spo0A and wherein the modified cell does not produce spores; (b) cultivating the transformed modified B. megaterium cell under conditions that permit expression of the nucleotide sequence to produce the heterologous protein; and (c) optionally isolating and/or purifying the heterologous protein.

In embodiments of this aspect of the invention, the modified B. megaterium cell is as described in any embodiment herein. In particular embodiments, the B. megaterium genome comprises a deletion of spo0A. In additional embodiments, the B. megaterium genome further comprises a deletion or functional deletion of the xylA and xylR genes and cannot metabolize xylose. In additional embodiments, the B. megaterium genome further comprises a deletion or functional deletion of one or more genes from the ilv-leu operon and cannot synthesize leucine. In still further embodiments, the B. megaterium genome comprises a deletion or functional deletion of the leuC and leuD genes. In particular embodiments, the leuC and leuD genes are deleted.

The invention also provides a B. megaterium expression system comprising (1) a modified asporogenous, leucine auxotrophic B. megaterium strain that comprises a deletion or functional deletion of spo0A, leuC and leuD in its genome and (2) an expression vector suitable for use with a B. megaterium cell that comprises a sequence of nucleotides that encodes leuC and a sequence of nucleotides that encodes leuD. In some embodiments, the leuC and leuD nucleotide sequences are operably linked to an ilv-leu promoter, or a variant ilv-leu promoter selected from the group consisting of: P503, P306, P201 and Pleu-1. In further embodiments, the B. megaterium strain comprises a deletion or functional deletion of xylA and xylR and cannot metabolize xylose.

All publications mentioned herein are incorporated by reference for the purpose of describing and disclosing methodologies and materials that might be used in connection with the present invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Bacterial Strains and Plasmids.

The integration vector, pCL10, was obtained from Chia Lee (Sau et al., Molecular Characterization and Transcriptional Analysis of Type 8 Capsule Genes in Staphylococcus aureus. J. Bacteriol. 179: 1614-1621 (1997)). The plasmid contains a temperature sensitive mutation to the origin of replication that prevents it from replicating at temperatures above 40° C. B. megaterium strain DSM319 was obtained from DSMZ (Leibniz Institute DSMZ, Braunschweig, Germany). B. megaterium expression vectors pMM1522, pHIS1522 and p3STOP1623 hp were obtained from Boca Scientific (Boca Raton, Fla., U.S. distributor for MoBiTec GmbH, Goettingen, Germany). DH10B competent E. coli were obtained from Invitrogen (Life Technologies Corp., Carlsbad, Calif.). Gene synthesis was performed by GenScript (GenScript USA, Inc., Piscataway, N.J.). WH320 protoplasts were obtained from Boca Scientific (Boca Raton, Fla., U.S. distributor for MoBiTec GmbH, Goettingen, Germany.

### Example 2

Spo0A Knockout Strain: spo0A Knockout Cassette in Integration Vector pCL10

A partial sequence of the B. megaterium spo0A was obtained from Genbank (Accession No. U09974). This sequence was aligned to the chromosomal sequence from strain QMB1551 (Quarter Master Bacterium #1551, ATCC12872, sequence available from Northern Illinois University) to obtain the complete sequence of the spo0A gene as well as 1000 bp regions upstream and downstream from the gene.

Primers were designed, based on the QMB1551 sequence, to amplify ˜500 bp regions flanking spo0A. A region of 453 bp upstream from spo0A was amplified with primers, spoF5 (5′ AAC CGG TTT GTC GAC GTT TCC GGC TAC TAA AGG AAT GG 3′, (SEQ ID NO:15)) and spo5R3 (5′ AAC CGG TTT GCA TGC CCT TTA TGT TAT ACG TCG TTT CC 3′, (SEQ ID NO:16)), which were designed with flanking SalI and SphI restriction sites. A downstream 457 bp region was amplified with primers spo3F5 (5′ AAC CGG TTT GCA TGC GGG GTT TTG AGT GAG CTG ATA CC 3′, (SEQ ID NO:17)) and spo3R3 (5′ AAC CGG TTT GAA TTC CGA TAT CTT CAG CTT AAT CGG AG 3′, (SEQ ID NO:18)), which were designed with flanking SphI and EcoRI sites. Bacillus megaterium strain DSM319 genomic DNA was used as a template for the spo0A flanking region amplification. DSM319 was also used to generate a genetically modified expression host.

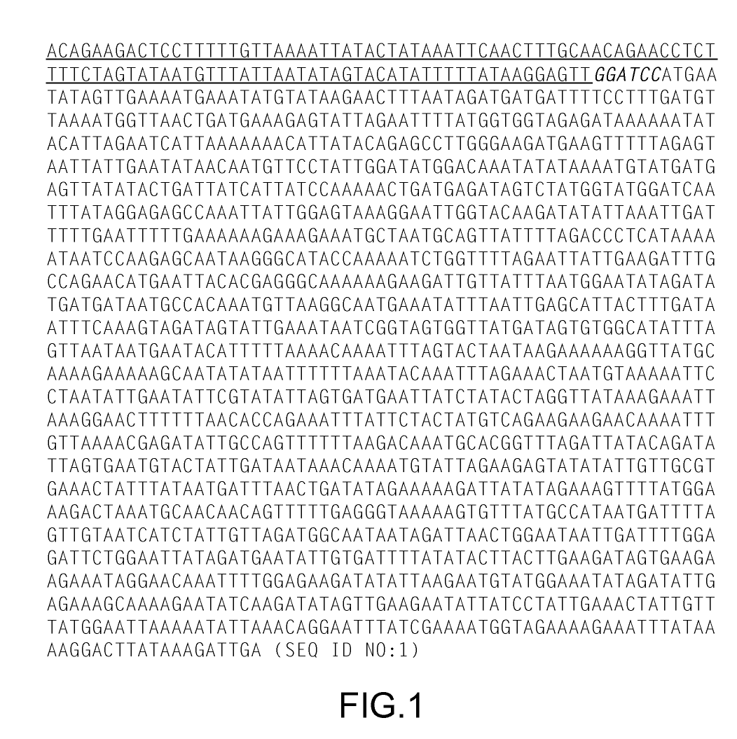

The resistance marker, aacA-aphD, (Genbank M18086, aminoglycoside resistance determinant of the transposon Tn4001), which confers gentamicin and kanamycin resistance, was inserted between the upstream and downstream flanking regions (Rouch et al., Journal of General Microbiology 133: 3039-3052 (1987), see FIG. 1 for nucleotide sequence). This marker was used to select for integration events. aacA-aphD expression was under the control of the Staphylococcus aureus constitutive dfrA promoter (Genbank NC_014369, Grkovic et al., Stable low-copy-number Staphylococcus aureus shuttle vectors. Microbiol. 149: 785-794 (2003)). PdfrA-aacA-aphD was amplified from the vector gent-TA, into which PdfrA-aacA-aphD had previously been cloned using primers gent-F (5′ AAC CGG TTT GCA TGC GGT ACC ACA GAA GAC TCC TTT TTG 3′, (SEQ ID NO:19)) and gent-R (5′ AAC CGG TTT GCA TGC TCA ATC TTT ATA AGT CCT TTT ATA AAT TTC 3′, (SEQ ID NO:20)) which contained flanking SphI restriction sites. The aac-aphD sequence was PCR amplified from S. epidermis strain 8751.

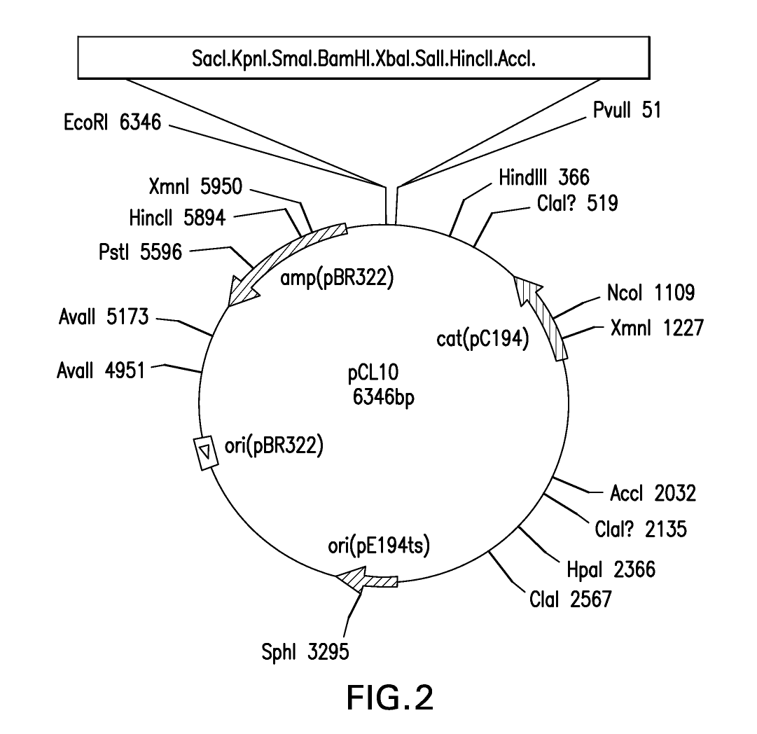

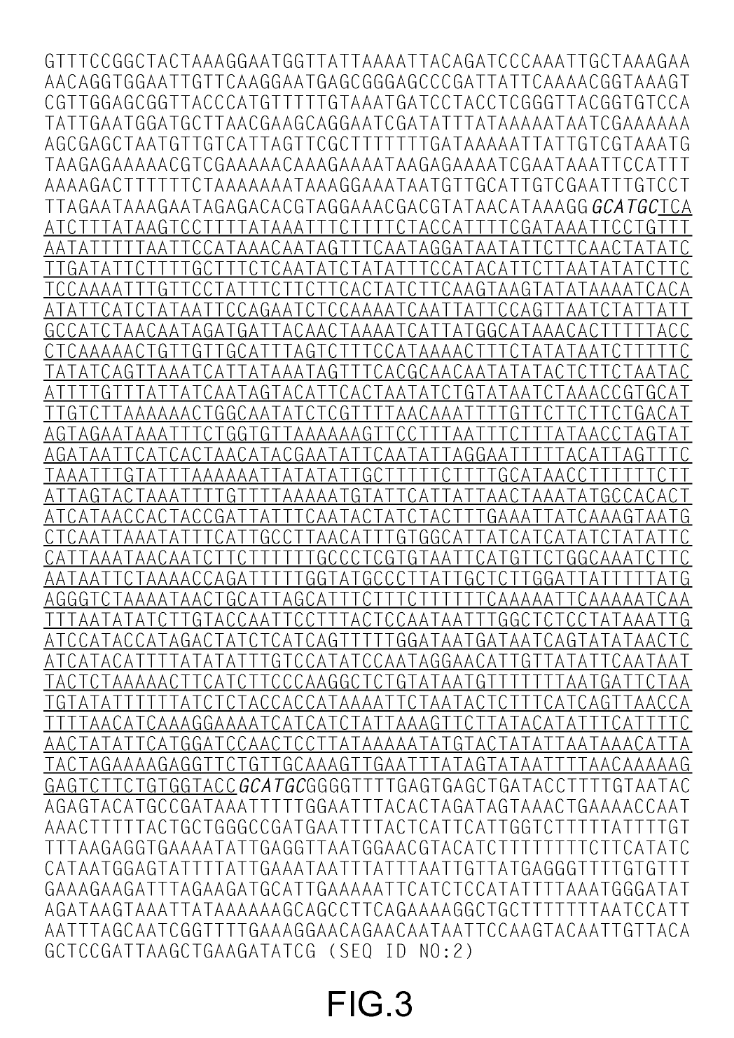

The spo0A upstream and downstream flanking regions were ligated into the gram positive integration vector pCL10 (see FIG. 2) using the restriction sites SalI and EcoRI. Next, the plasmid was digested with SphI to insert PdfrA-aacA-aphD between the spo0A flanking regions (see FIG. 3). The resulting plasmid was called spo0A gent pCL10.

B. megaterium DSM319 protoplasts were prepared following the procedure in Barg et al. (Protein and vitamin production in Bacillus megaterium. Methods in Microbiology, 18: 205-223 (2005)). spo0A gent pCL10 was transformed into the DSM319 protoplasts following the procedure in MoBiTec's Bacillus megaterium Protein Production Manual. Transformants were grown overnight at 30° C. on LB agar plates containing 15 ng/ml chloramphenicol.

### Example 3

### Integration Protocol

Homologous recombination was used to generate a spo0A deletion strain. Individual colonies were picked and grown for three days at 30° C. in LB media containing 30 μg/mlkanamycin, with daily subculturing (1:1000 dilution of the culture in fresh media). Next, the cultures were diluted 1:1000 in LB without antibiotic selection and grown for 2 days at 42° C., with daily subculturing, for plasmid curing. Finally, the cultures were diluted 1:100 and spread on LB agar plates containing 30 ng/mlkanamycin to screen for integration.

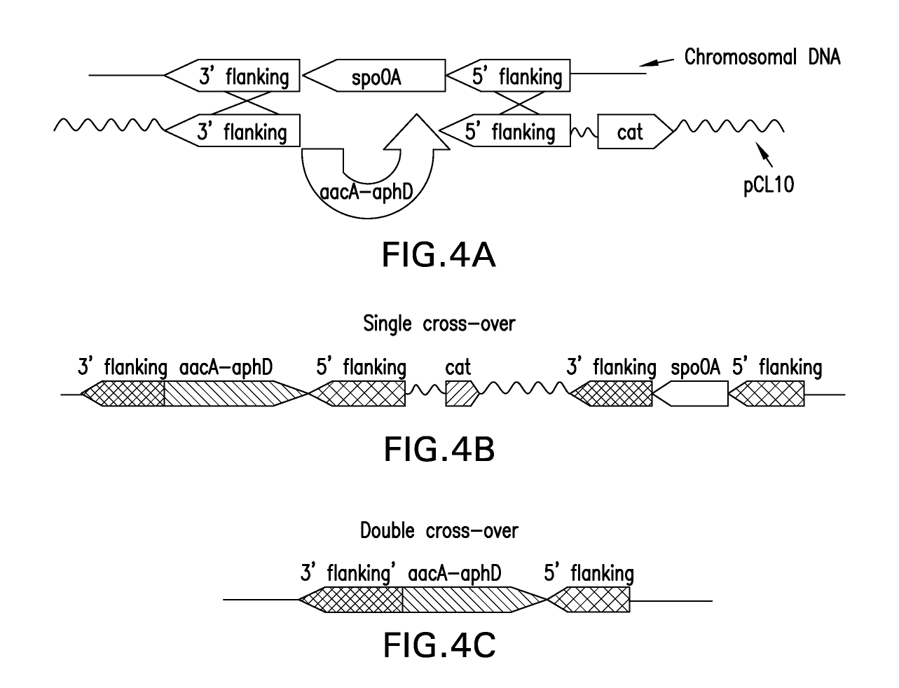

Possible outcomes from the integration protocol are as follows: (a) the plasmid is maintained by the bacteria, (b) a single crossover event occurs, the entire plasmid is integrated into the chromosomal DNA or (c) a double crossover event occurs and only the knockout cassette is integrated (see FIG. 4).

Initially, bacteria were screened for integration by their antibiotic resistance profile. pCL10 carries a resistance marker for chloramphenicol and the knockout cassette carries resistance to kanamycin. If the plasmid is maintained or a single cross over event occurs, the bacteria will be resistant to both kanamycin and chloramphenicol. If the desired double crossover event occurs, the bacteria will maintain kanamycin resistance but lose chloramphenicol resistance. Four isolates were found that met the desired criteria.

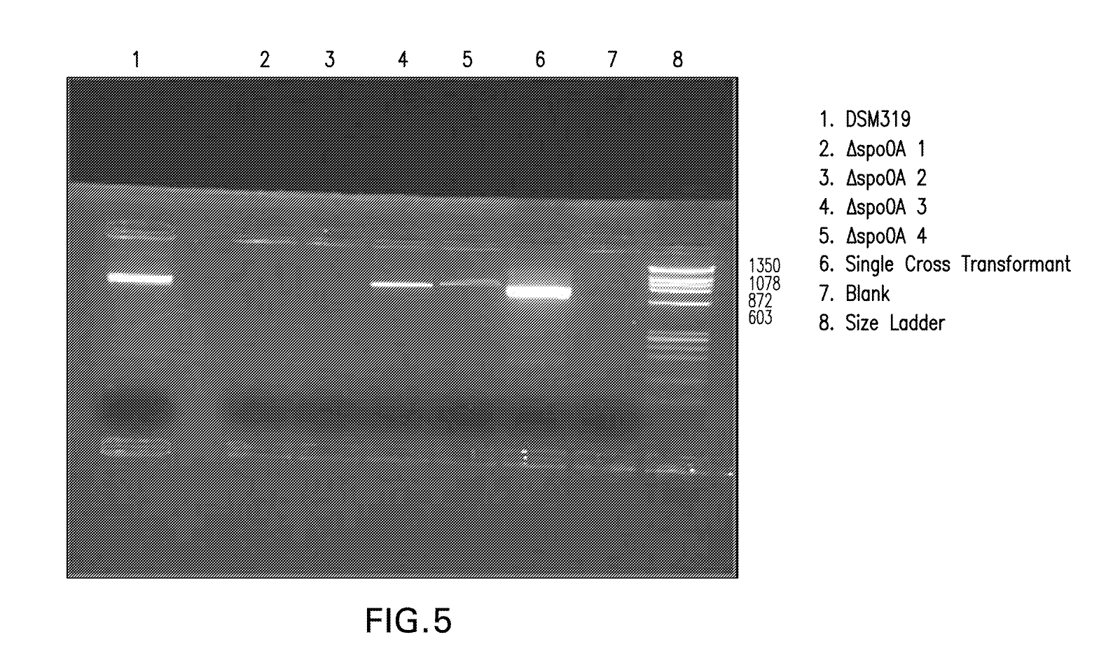

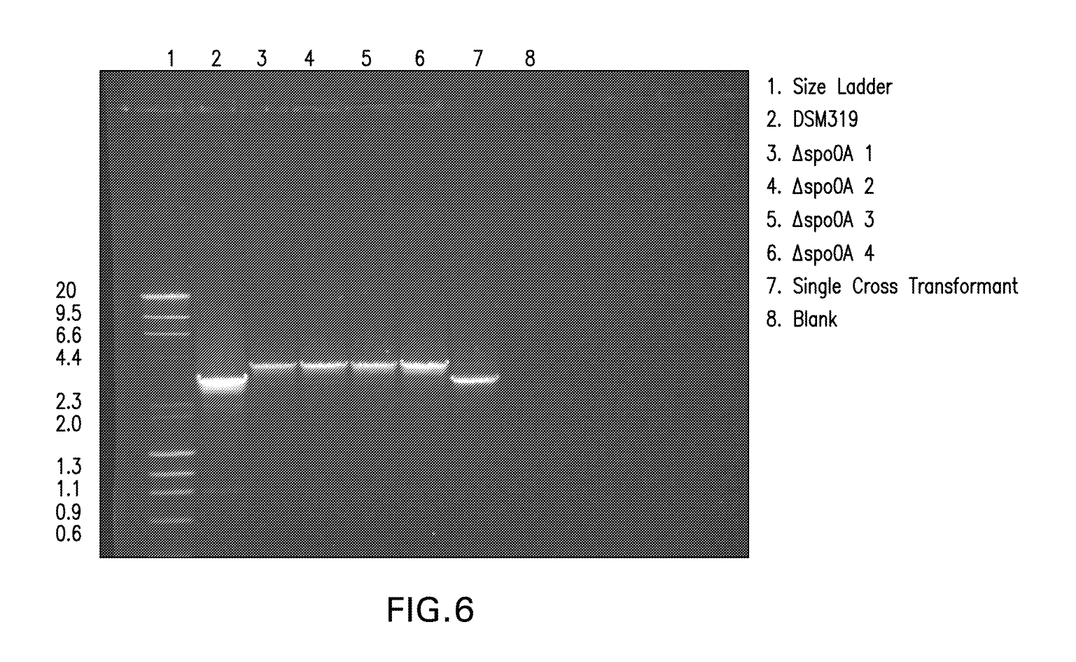

Chromosomal DNA was isolated from these four isolates using Promega's Wizard® Genomic Purification Kit (Promega Corp., Madison Wis.). Colonies were screened by 2 PCR amplifications. In the first, oligonucleotide primers were designed to amplify the spo0A gene (788 bp, spoOA 5′ GTA TGC TTG GTT GAT GAT AAT CGG G 3′ (SEQ ID NO:21) and spoOA-R 5′ GCA GTC CTA TCC CTT TCA ACT CGC 3′ (SEQ ID NO:22)). PCR products were run on a 1% agarose gel and visualized with ethidium bromide. Desired isolates (named ΔspoOA 1 and Δspo0A 2) were negative for this amplification (FIG. 5). Next, PCR primers were designed to the upstream and downstream region flanking the knock out cassette (spo-SPCR 5′ CGA AAT TGC GGG TAT TCA AGT TGG AG 3′(SEQ ID NO:23) and spo-3RPCR 5′ GCA TTT TTC GCA CCG TTA GTT GTT GG 3′ (SEQ ID NO:24)). These primers were designed to amplify the chromosomal DNA and shared no homology with the plasmid. Since the nucleotide sequence of the aacA-aphD resistance marker is about 700 bp longer than that of the spo0A gene, a successful double cross-over would result in an amplification product that is 700 bp longer than the PCR product resulting from chromosomal DNA (3.8 kb for isolates with double cross-over v. 3.1 kb for wild-type DNA, FIG. 6). PCR products were run on a 1% agarose gel and visualized with ethidium bromide. Sizes of PCR amplification products from all four isolates (ΔspoOA-1−ΔspoOA-4) were consistent with a double cross-over event. Based on the results of the two gels, it appeared that AspoOA1 and AspoOA2 had the desired cross-over event and the integration plasmid had been cured. ΔspoOA3 and ΔspoOA4 had integrated that plasmid but also had maintained a functional copy of the gene probably due to maintaining the plasmid.

A final screen was conducted that consisted of heat inactivation of vegetative growth. Bacteria were grown on BD BBL™ AK agar #2 (sporulating agar, Becton Dickinson and Co., Franklin Lakes, N.J.) for 3 days. 25 OD units of bacteria were transferred to 1 mL PBS. Bacteria were heat inactivated at 85° C. for 30 minutes. The heat was used to kill vegetative growth and allow for the initiation of germination of spores. Following heat treatment, 100 μl of undiluted and serially diluted bacteria were spread on LB plates and incubated overnight at 37° C. Two isolates were found to have the spo0A deletion, Δspo0A1 and Δspo0A2. A second round of 42° C. curing was performed to insure plasmid curing. Two isolates from the second round of curing were rescreened and found to be correct. They were named Δspo0A-A and Δspo0A-B.

### Example 4

Expression of Nap_3 mTcdB in Asporogenous B. megaterium Strain

Codon optimization of the Clostridium difficile toxin B (TcdB) sequence from the NAP 1 strain was performed by Genscript (Piscataway, N.J.). The sequence contained three mutations in the glucosyltransferase domain to reduce toxicity (W102A, D288A, E515Q). The optimized sequence was cloned in the B. megaterium expression vector pHIS1522 (Boca Scientific, Boca Raton, Fla.) to create plasmid nap_3 mTcdB_B1 (see Heinrichs et al., WO 2013/112867). Protoplasts were prepared for asporogenous strain Δspo0A-A following the procedure in Barg et al., 2005, supra, and transformed following the procedure in MoBiTec's Bacillus megaterium Protein Production Manual. The nap_3 mTcdB plasmid was also transformed into the sporulating strain WH320 (MoBiTec) for use as a control.

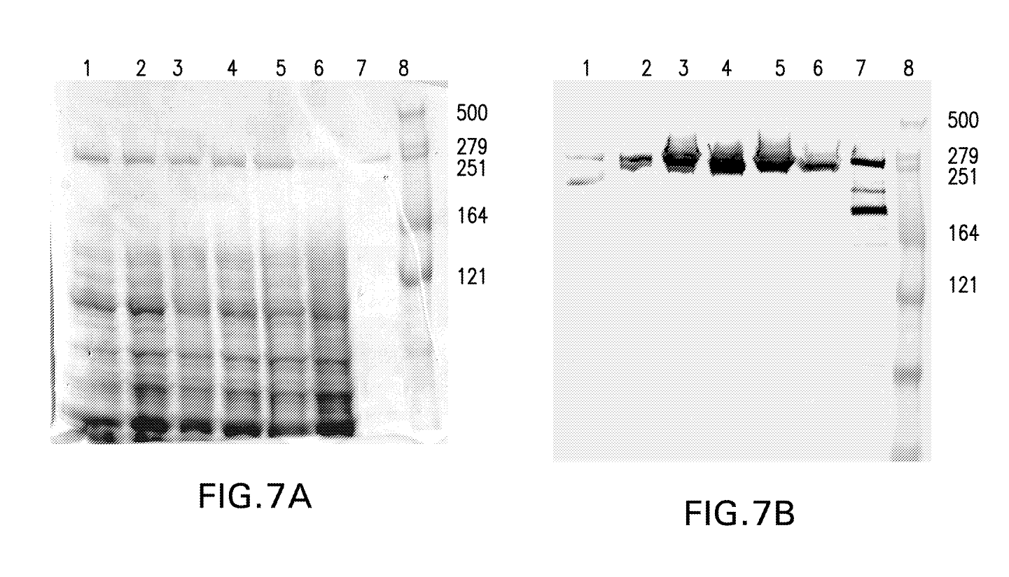

For expression studies, five individual colonies from the strain Δspo0A-A and one from WH320 were grown overnight in LB-15 μg/ml tetracycline. The next morning, 25 mL production cultures in LB-15 μg/ml tetracycline were inoculated with 3 mL of the seed cultures and grown to a concentration of OD600=0.40-0.50. Expression was induced with 0.5% xylose for 18 hours. Cell paste was harvested by centrifugation and stored at −80° C. until it was analyzed. Cell lysis was performed using BugBuster® reagent (Novagen Inc., Merck KGaA, Darmstadt, Germany) containing 200 μg/mL lysozyme (Sigma-Aldrich, St. Louis, Mo.) for 45 minutes at room temperature. Gel electrophoresis and Western blotting were performed following standard techniques. The Western blot was developed using the anti-TcdB mAb 5A8E11 (Novus Biologicals, Littleton, Colo.). Analysis revealed all Δspo0A-A colonies expressed nap_3 mTcdB, most at higher levels than WH320 (FIG. 7).

### Example 5

xylA/R Knockout Strain

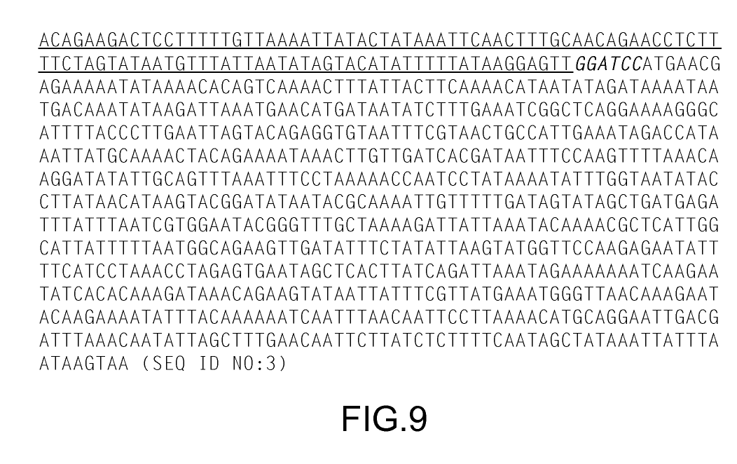

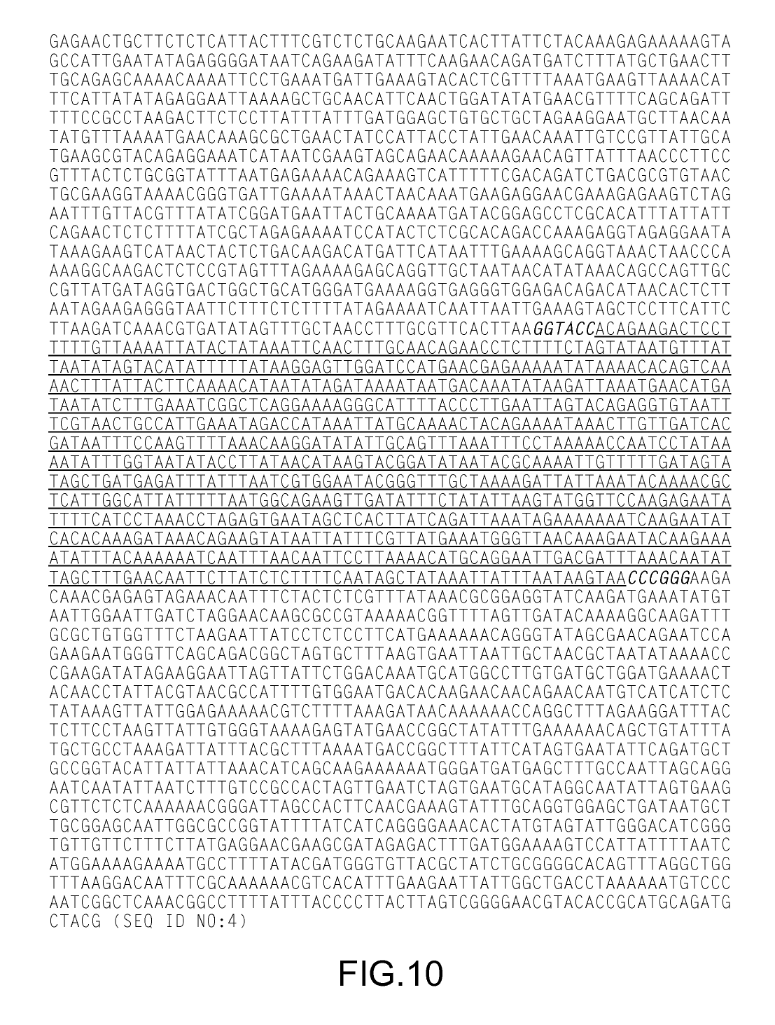

Next, the genes xylA and xylR were deleted from the chromosomal DNA of the sporulation deficient strain Δspo0A-A (FIG. 8). The integration plasmid, pCL10, was used to generate the xylAR deletion strain. The sequence for the xylose utilization operon for B. megaterium strain DSM319 was taken from Genbank (CP001982). Two pairs of primers were designed to amplify 1114 base pairs downstream from xylA with flanking restriction sites SmaI and BamHI (xylA-F 5′ AAC CGG TTT CCC GGG AAG ACA AAC GAG AGT AGA AAC (SEQ ID NO:25) and xylB-R2 5′ AAC CGG TTT GGA TCC CGT AGC ATC TGC ATG CGG TGT ACG 3′ (SEQ ID NO:26)) and a region 1021 base pairs downstream from xylR with flanking SacI, SmaI and KpnI restriction sites (xyl-F1 5′ AAC CGG TTT GAG CTC GCG AAC TGC TTC TCT CAT TAC 3′(SEQ ID NO:27) and xylR-sma-kpn 5′ TTC CCG GGA CGT ACG GTA CCT TAA GTG AAC GCA AAG GTT AGC AAA C 3′ (SEQ ID NO:28)). These flanking regions were ligated into pCL10 flanking the erythromycin resistance marker ermC (Genbank M17990, FIG. 9, Projan et al., Replication Properties of pIM13, a Naturally Occurring Plasmid Found in Bacillus subtilis, and of Its Close Relative pE5, a Plasmid Native to Staphylococcus aureus. J. Bacteriol. 169(11):5131-5139 (1987)). The sequence of ermC was obtained from Genbank and the gene was synthesized by Genscript with flanking SmaI and KpnI restriction sites (Piscataway, N.J.). Expression of ermC was again controlled by the dfrA promoter. The xylAR pCL10 integration plasmid was named xylAR_erm1. The nucleotide sequence of the xylAR knockout cassette is provided in FIG. 10 (SEQ ID NO:4).

### Example 6

Integration of xylA/R Knockout Cassette

Protoplasts were made for the asporogenous Bacillus megaterium strain Δspo0A-A following the procedure in Barg et al. (2005, supra). Transformations were performed following the procedure in MoBiTec's Bacillus megaterium Protein Production System Manual. Transformants were grown overnight at 30° C. on tryptic soy agar (TSA) plates containing 50 μg/ml erythromycin (Teknova, Hollister, Calif.). Individual colonies were picked and grown for three days at 30° C. in LB containing 50 μg/ml erythromycin, with daily subculturing (1:1000 dilution of the culture in fresh media). Next, the cultures were diluted 1:1000 in LB media without antibiotic selection and grown for 2 days at 42° C., with daily subculturing, for plasmid curing. The cultures were diluted 1:100 and spread on TSA plates containing 50 μg/ml erythromycin.

### Example 7

### Screening for Integration Events

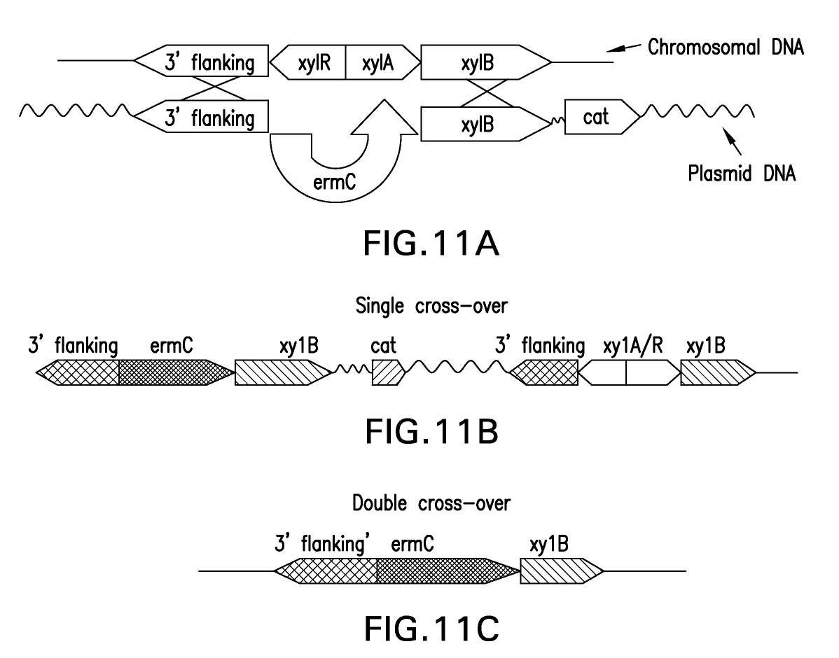

Initially, bacteria were screened for integration by their antibiotic resistance profile (FIG. 11). pCL10 carries a resistance marker for chloramphenicol and the knockout cassette carries resistance to erythromycin. Isolates were selected that were resistant for erythromycin and sensitive to chloramphenicol. Twelve isolates were found that had the correct antibiotic resistance profile. They were named Δspo0ΔAxylAR 1-12.

The twelve isolates were screened for their ability to metabolize xylose. Isolates were streaked on LB agar plates with glucose and on M9 minimal agar plates with xylose as the sole carbon source. Isolates were only able to grow on LB agar plates and not on the M9 plates; thus, all twelve xylAR knockouts were unable to metabolize xylose.

### Example 8

Expression of Nap_5 mTcdB in a ΔSpo0ΔAxylAR B. megaterium Strain

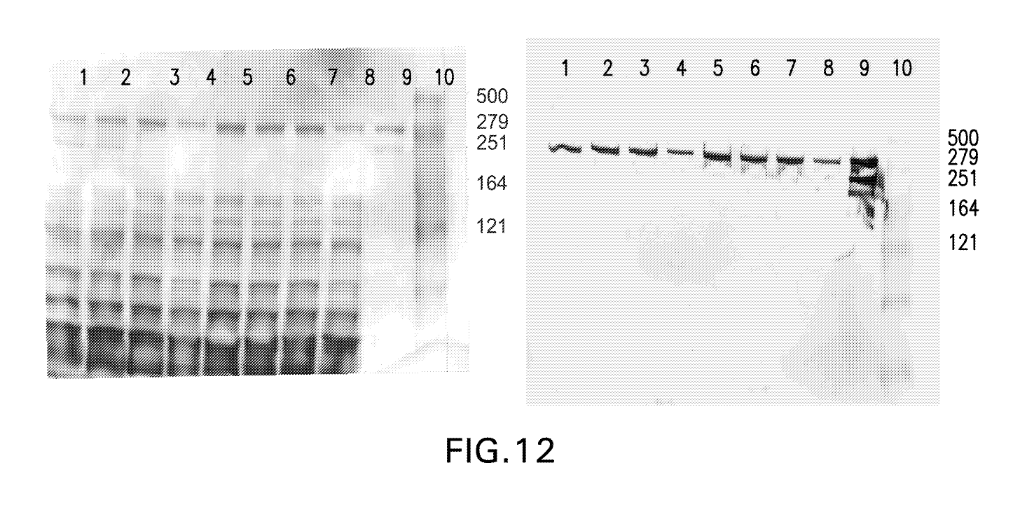

Expression plasmid nap_3 mTcdB_B1 used in the Δspo0A expression studies was modified with two additional mutations: C698A and W520A. Mutations were introduced using Stratagene's QuikChange™ Site Directed Mutagenesis Kit (Agilent Technologies, Santa Clara, Calif.). Protoplasts were prepared for asporogenous strain ΔxylAR 1 following the procedure in Barg et al. (2005, supra) and transformed following the procedure in MoBiTec's Bacillus megaterium Protein Production Manual. For expression studies, eight individual colonies were grown overnight in LB-15 μg/ml tetracycline. The next morning, 25 ml production cultures in LB-15 μg/ml tetracycline were inoculated with 3 ml of the seed cultures and grown to a concentration of OD600=0.40-0.50. Expression was induced with 0.5% xylose for 18 hours. Cell paste was harvested by centrifugation and stored at −80° C. until it was analyzed. Cell lysis was performed using BugBuster® reagent (Novagen Inc., Merck KGaA, Darmstadt, Germany) containing 200 μg/ml lysozyme for 45 minutes at room temperature. Gel electrophoresis and Western blotting were performed following standard techniques. The Western blot was developed using the anti-TcdB mAb 5A8-E11 (Novus Biologicals, Littleton, Colo., FIG. 12).

### Example 9

### Generation of a Leucine Auxotrophic Strain

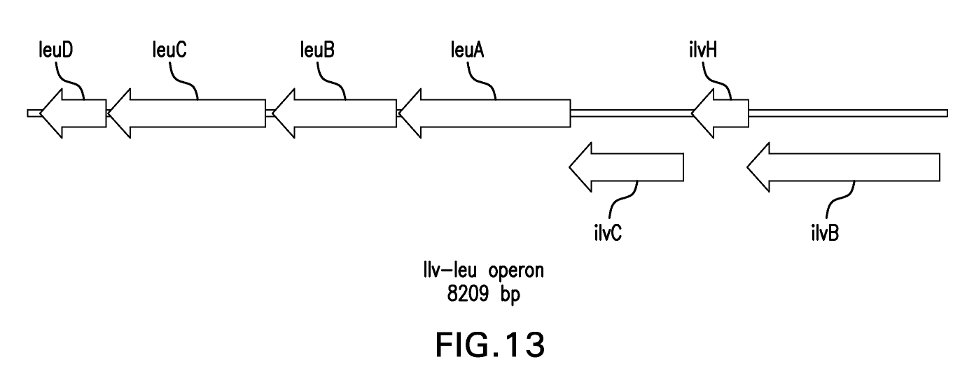

The sequence of the ilv-leu operon from B. megaterium strain DSM319 was obtained from Genbank. We selected the genes leuC and leuD for deletion due to their being the most distal genes in the operon (FIG. 13). It was also thought that since leuC and leuD encode the two subunits of the 3-isopropylmalate dehydratase, these genes should be co-expressed in the modified B. megaterium expression system; thus they were deleted from the B. megaterium strain and added to the complementation plasmid (described, infra).

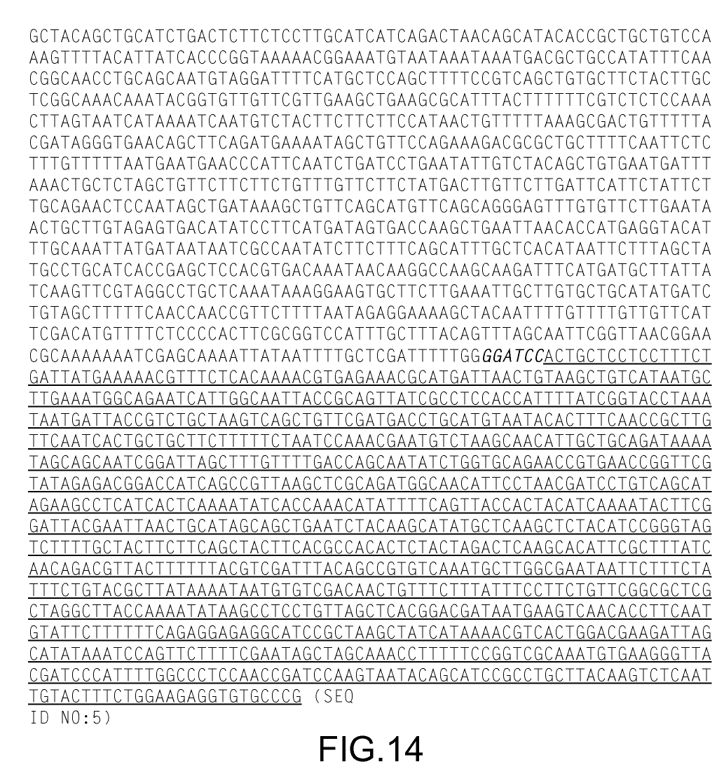

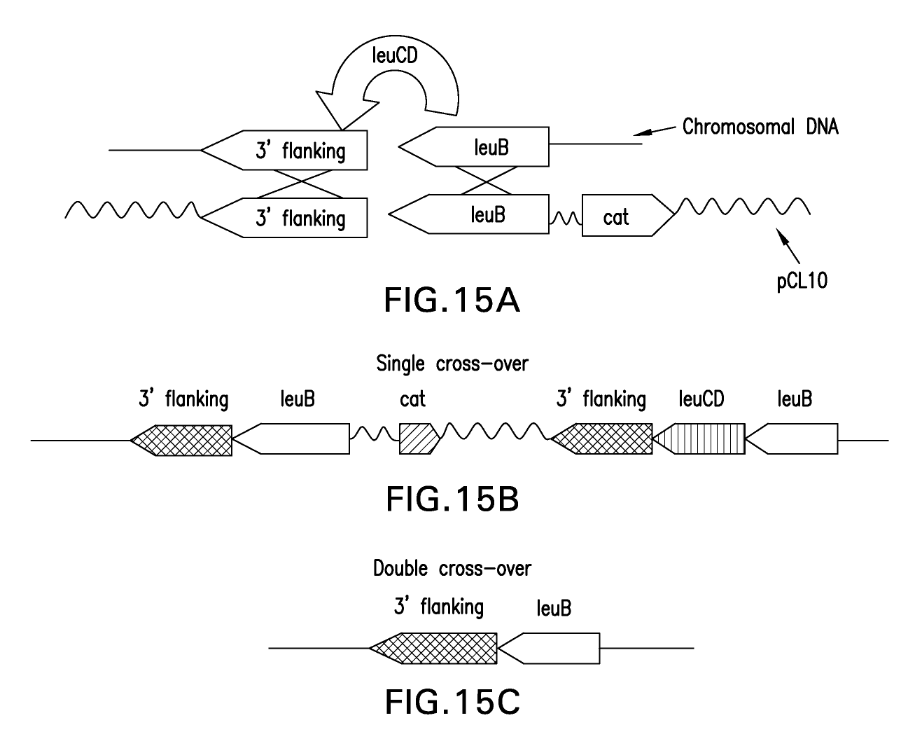

The knockout cassette was set up in the integration vector pCL10. The knockout cassette consisted of a 1017 bp fragment at the 3′ region of the leuB and a 1018 bp fragment at the region downstream from leuD (FIG. 14). The fragments were PCR-amplified using the primer pairs leuB-F (5′AAC CGG TTT GGA TCC ACT GCT CCT CCT TTC TGA TTA TG 3′ (SEQ ID NO:29))/leuB-R (5′AAC CGG TTT CCC GGG CACA CC TCT TCC AGA AAG TAC 3′ (SEQ ID NO:30)) with flanking restriction sites BamHI (forward primer) and XmaI (reverse primer) and leuD-F (5′ AAC CGG TTT TCT AGA GCT ACA GCT GCA TCT GAC TCT TC 3′ (SEQ ID NO:31))/leuD-R (5′ AAC CGG TTT GGA TCC CCA AAA ATC GAG CAA AAT TAT AA 3′ (SEQ ID NO:32)) containing flanking restriction sites XbaI and BamHI (Sigma-Aldrich, St. Louis, Mo.). In this case, an antibiotic resistance marker was not inserted between the upstream and downstream flanking sequence since leucine biosynthesis rather than antibiotic resistance was used to screen for integration. Protoplast transformation followed the procedure previously listed. Protoplasts were generated from B. megaterium strain Δspo0AΔxylAR1 generated above. The integration protocol followed the procedure previously listed. Following plasmid curing, bacteria were spread on LB plates. Individual colonies were streaked on M9 minimal media agar plates and LB plates (Teknova, Hollister, Calif.). Isolates were screened by their ability to grow on M9 minimal media agar plates. Since the desired leucine auxotrophs were not able to biosynthesize leucine, they were unable to grow on M9 minimal media agar plates (FIG. 15). Four leucine auxotrophic isolates were found and named Δspo0AΔxylARΔleuCD A-D.

### Example 10

Design of LeuCD complementation expression plasmids, promoters and LeuCD constructs

The second half of leucine complementation involved moving the leuC and leuD genes to the expression plasmid. The genes remained under the control of the ilv-leu promoter.

The ilv-leu promoter was attenuated to prevent the copy number of complementation expression plasmids in B. megaterium from being affected by ilv-leu promoter strength. This strategy was based on previous work with a S. cerevisiae expression system utilizing Leu-2 complementation for selection/plasmid maintenance, in which a mutation in the Leu-2 promoter both reduced expression of Leu-2 (less than 5% of that observed with the wild type sequence) and conferred a strong selective advantage on cells that contain a high copy number of the expression plasmid (Erhart et al., The presence of a defective leu2 gene on 2μ DNA recombinant plasmids of Saccharomyces cerevisiae is responsible for curing and high copy number. J. Bacteriol. 156(2) 625-635 (1983); Pronk, J., Auxotrophic yeast strains in fundamental and applied research. Applied and Environmental Microbiology. 68(5) 2095-2100 (2002)).

Grandoni et al. (J. Bacteriol. 175(23) 7581-7593 (1993)) investigated regulation of the Bacillus subtilis ilv-leu operon. Expression of this operon is regulated in part by transcriptional attenuation. The leader mRNA contains a series of four inverted repeats (stem and loop structures) that are responsible for the leucine dependent regulation of the operon (Grandoni et al., Transcriptional regulation of the ilv-leu operon of Bacillus subtilis. J. Bacteriol. 174(10)3212-3219 (1992), Grandoni et al., 1993, supra). They investigated a series of deletional mutations to the stem and loop structures to determine the effect the mutations would have on β-galactosidase-specific activity (Grandoni at al., 1993, supra). We identified three mutant leader regions for further investigation: constructs 201, 306, and 503. The leader sequence for construct 201 was reported to produce 550-fold lower activity than with the wild type sequence. Expression for construct 306 was reported to be about 60-fold lower than wild-type and expression for construct 503 was about 5-fold lower. Grandoni et al., 1993, supra.

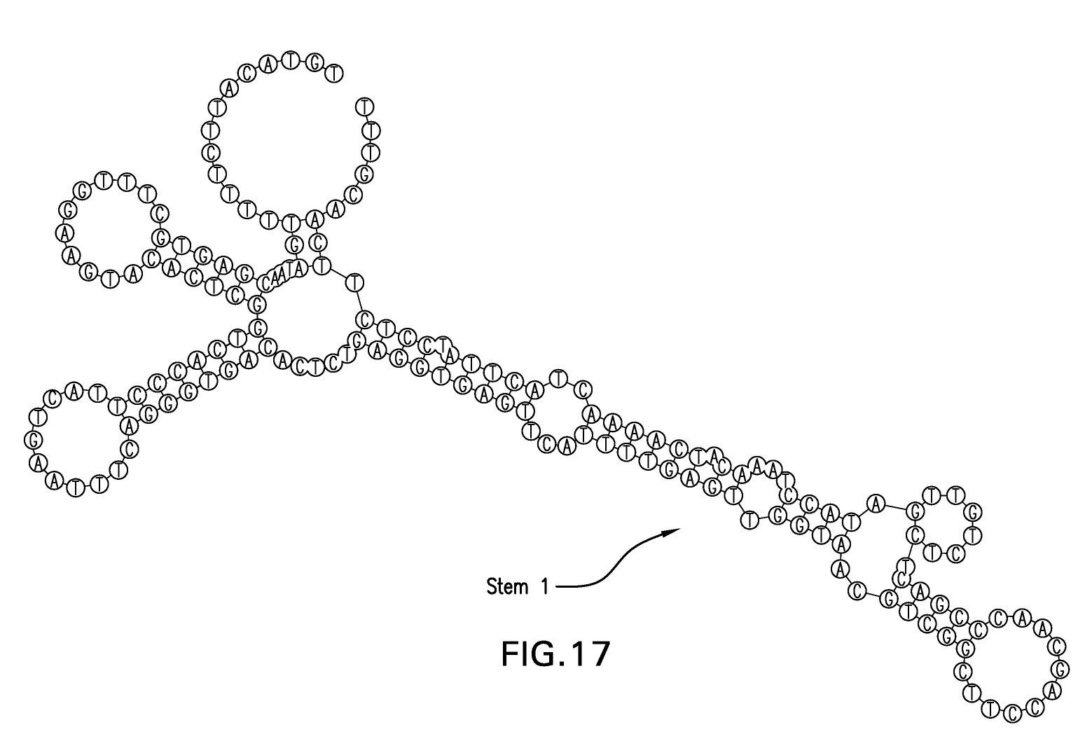

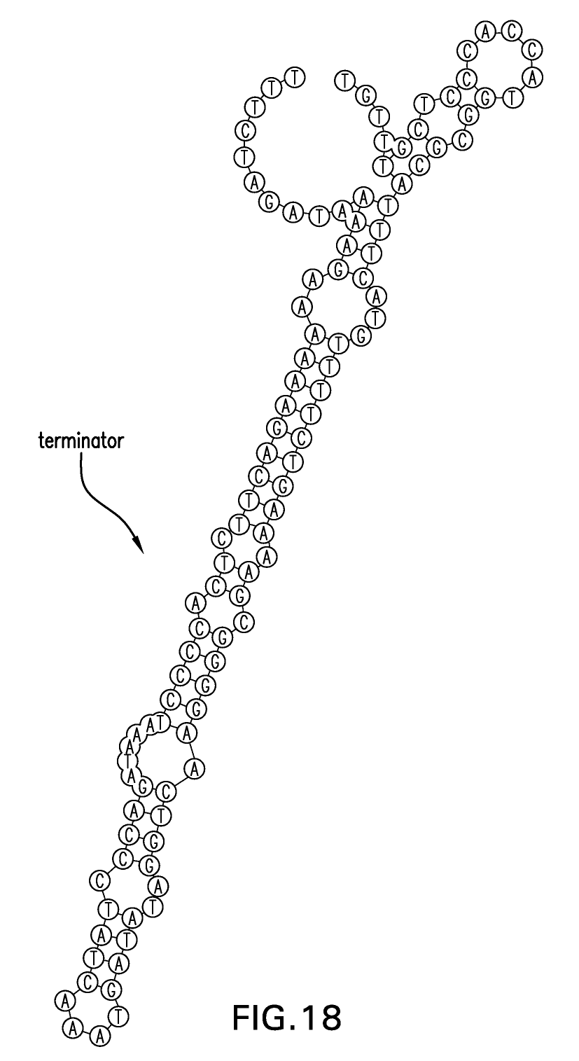

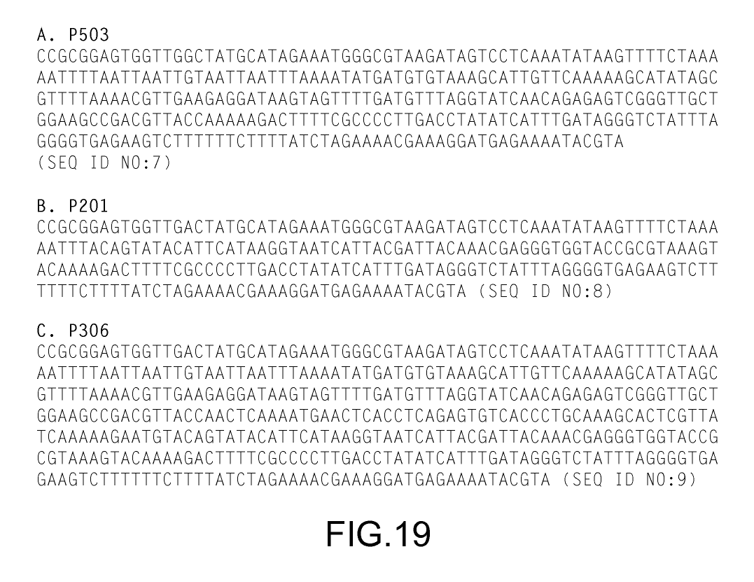

The ilv-leu promoter in B. megaterium has not been characterized. The B. subtilis ilv-leu promoter (Genbank L03181) was aligned to the region upstream from B. megaterium ilvB gene to identify a probable region for the promoter (SEQ ID NO:6, FIG. 16). Sequence homology was also used to predict the location of the −35 and -10 regions of the promoter. The locations of the four stem loop structures in the ilv-leu leader region were predicted using the web site RNA Fold (Institute for Theoretical Chemistry, University of Vienna; Vienna, Austria). Examples of the predicted structures are shown in FIGS. 17 and 18. This structural analysis was then used to try to generate deletions similar to those reported for B. subtilis, for promoters 201, 306 and 503 in the predicted B. megaterium ilv-leu promoter. Based on the RNAfold analysis of the promoter sequence, three mutant promoters were designed: P503, P306, and P201 (see FIG. 19 for nucleotide sequences of variant promoters). The mutant promoters include the following deletions: For P201, a 233 bp region between the predicted site +26 of the transcription start site (predicted from the location of the predicted −10 region) and the predicted protector (part of stem and loop structure) was deleted from the leader sequence. For P306, 30 bp were deleted from a region midway between the predicted Stem I and protector. Finally for P503, 153 bp were deleted from approximate 20 bp downstream from the 3′ end of Stem I to the terminator, deleting the predicted protector and antiterminator.

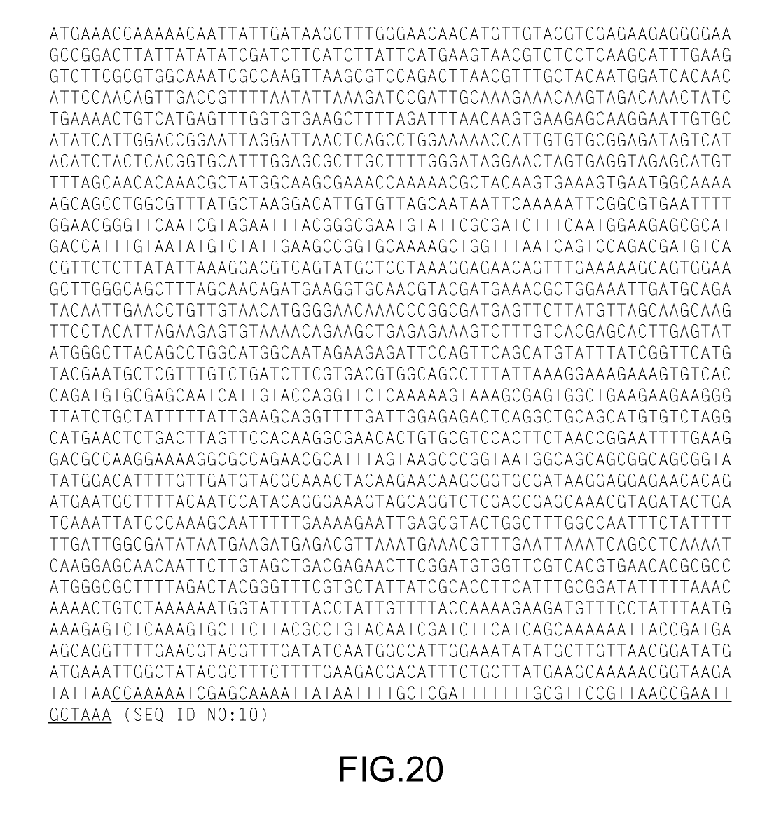

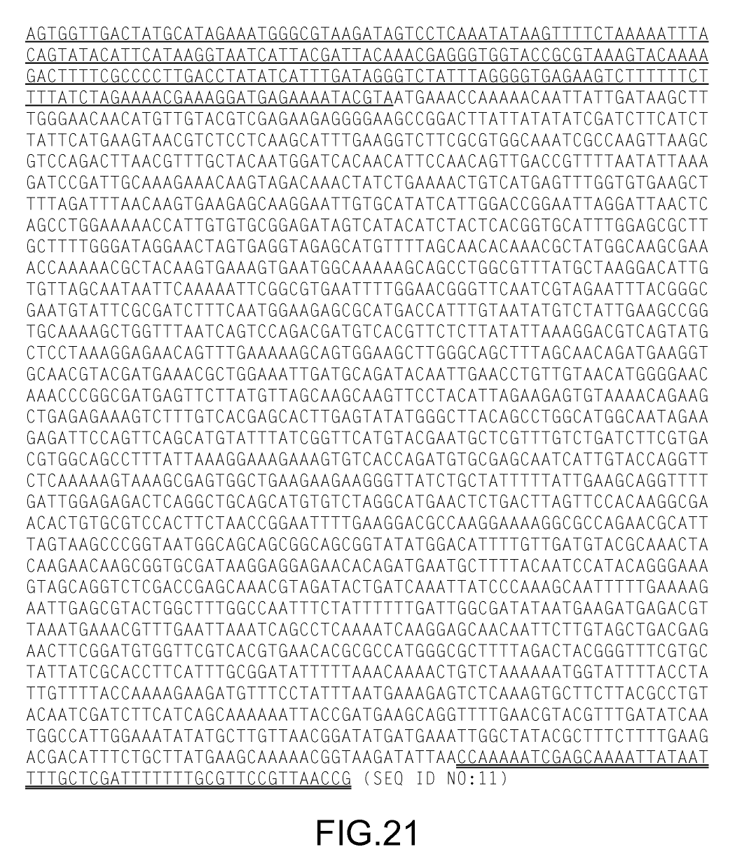

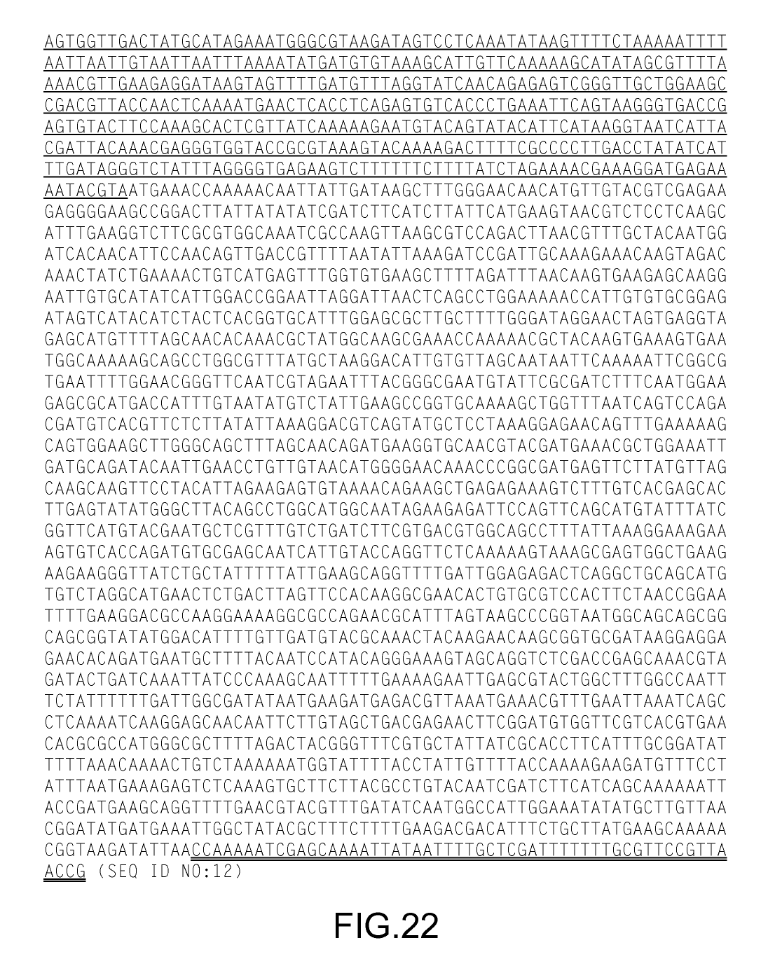

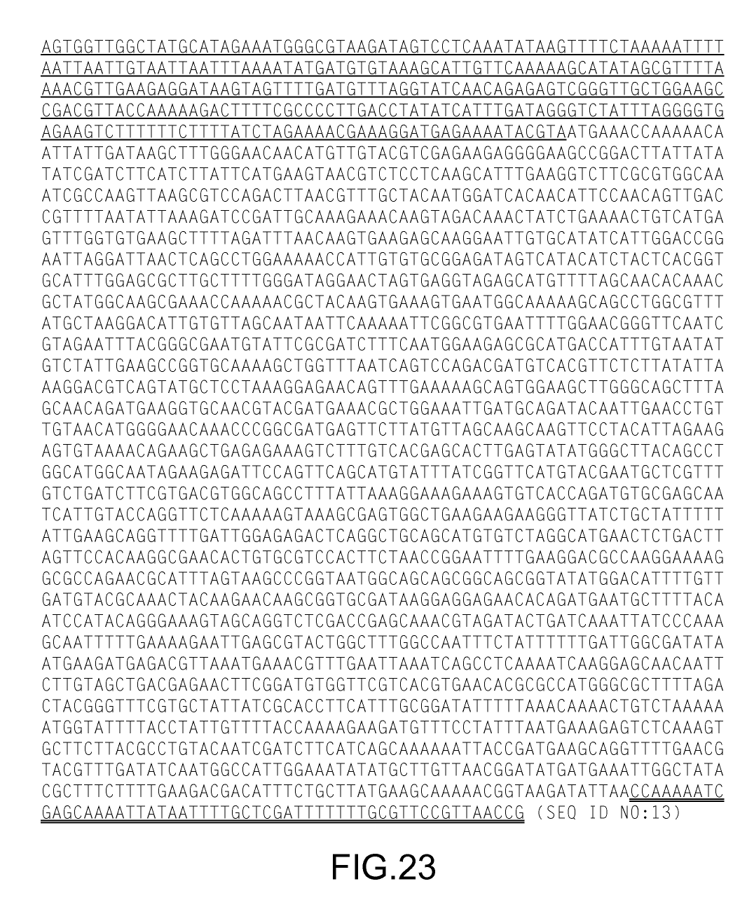

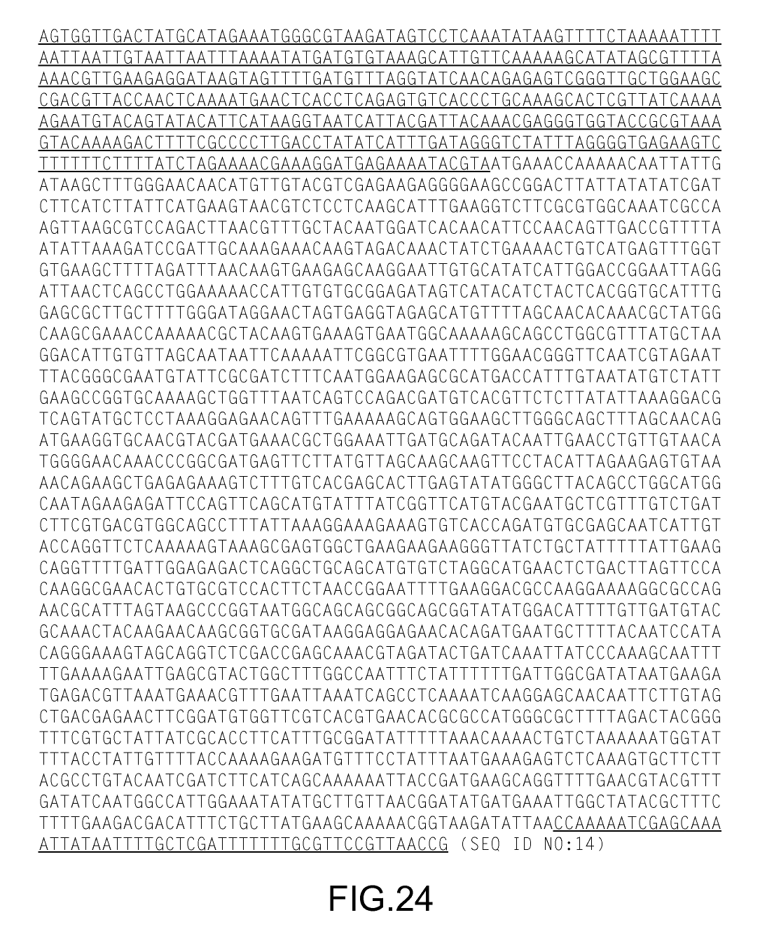

Four constructs were designed with different promoters, as discussed above. Each of the constructs comprised an ilv-leu promoter ((1) Pleu (wild-type promoter), (2) P201 (variant promoter), (3) P306 (variant promoter), or (4) P503 (variant promoter)), the wild type leuC and leuD genes (referred to as leuCD) and a transcriptional terminator. The sequence of leuCD was obtained from the genomic sequence of B. megaterium strain DSM319 (Genbank CP001982). An additional 65 base pairs downstream from leuD were included to incorporate a predicted transcriptional terminator (SEQ ID NO:10, FIG. 20). Gene synthesis of constructs Pleu, P201, P306, and P503 was performed by Genscript (Piscataway, N.J.). Constructs also contained flanking SacII and NheI restriction sites. The nucleotide sequence of the four constructs is shown in FIGS. 21-24 (SEQ ID NO's 11-14).

### Example 11

### LeuCD Complementation Plasmids

Two sets of vectors were created: (1) the tetracycline resistance marker was replaced with the leuCD cassette and (2) the leuCD cassette was inserted downstream from the tetracycline resistance marker. In set (2), tetracycline resistance or leucine biosynthesis was used for selection and plasmid maintenance.

Three different vectors were modified as described above: pMM1522, pHIS1522 and p3STOP1623 hp (MoBiTec GmbH, Goettingen, Germany). Pleuleu, P306leu and P503leu leuCD constructs were initially included in the study; however, we decided to focus on the constructs with the modified promoters (P306leu and P503leu) since the 469 bp ilv-leu promoter is on both the plasmid and in the chromosomal DNA. Sections of the leader sequence were deleted from the wild type sequence to create the modified promoters. These deletions should reduce the chance/risk of homologous recombination between the expression plasmid and chromosomal DNA.

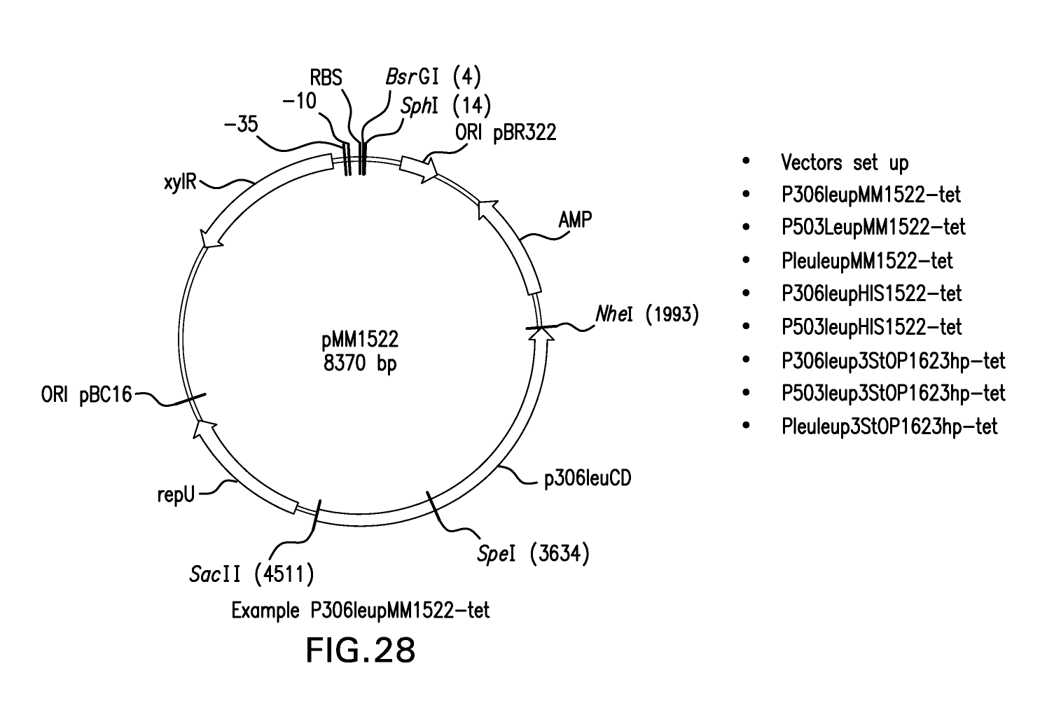

Site directed mutagenesis using Stratagene's QuikChange® Lightning Multi Site-Directed Mutagenesis Kit was used to mutate the BsrGI sites in the ilv-leu promoter (primer PleuBsrGI-F 5′ GCA CTC GTT ATC AAA AAG AAA GTA CAG TAT ACA TTC ATA AGG 3′ (SEQ ID NO:33)) and leuD (primer leuDBsrGI-F 5′ GCT TTC TTA CGC CTG TAC GAT CGA TCT TCA TCA GC 3′ (SEQ ID NO:34)). For the vectors in which the tetracycline resistance marker was replaced by leuCD cassettes, site directed mutagenesis was performed to add flanking SacII (primer SacII downstream 5′ GTG CCA CCT GAC CCG CGG GTC TAA GAA ACC 3′ (SEQ ID NO:35)) and NheI (primer NheI-upstream 5′-GAA CCC TGT TAC ATG CTA GCT CAT TAC ACT TC 3′ (SEQ ID NO:36)) restriction sites around the tetracycline resistance marker. For vectors pMM1522 and pHIS1522, which contain a NheI site in the backbone, mutagenesis was performed to remove this restriction site (primer pMM-NheI 5′ GCA TCG CCA GTC ACT ATG GCG TGC TGC AAG CGC TAT ATG CGT TGA TGC 3′ (SEQ ID NO:37)), as well as a SpeI restriction site and start codon from the multiple cloning site (primer Stop_Spe-F 5′ CAA AGG GGG AAA TGT ACA TTC GAA GAT CTC CGG AGC 3′ (SEQ ID NO:38)). A SpeI site is also present in the leuCD cassettes. After the mutagenesis was complete, standard molecular biology procedures were used to perform a NheI and SacII restriction digest of the three modified expression vectors (pMM1522, pHIS1522 and p3STOP1623 hp) and the modified Pleuleu constructs (Pleuleu, P306leu and P503leu). The leuCD cassettes were ligated into the expression vectors and transformed into DH10B competent E. coli (Invitrogen). Vectors were screened by restriction digest and DNA sequencing. A representative vector map for P306leupMM1522-tet, as well as a list of alternate vectors described herein in which the tetracycline resistance marker is replaced by leuCD cassettes, is provided in FIG. 28.

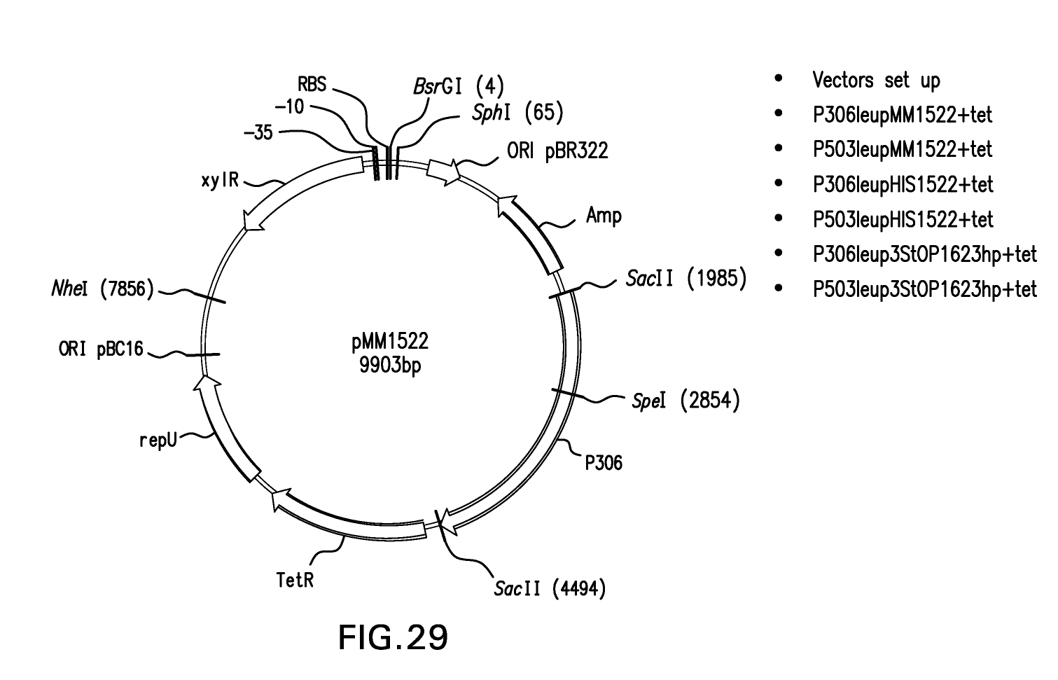

A second set of vectors were also constructed which maintain the tetracycline resistance marker. Again, the expression vectors selected for this study were pMM1522, pHIS1522 and p3STOP1623 hp. For these vectors, the P306leu and P503leu cassettes were used. Site directed mutagenesis using Stratagene's QuikChange® Lightning Multi Site-Directed Mutagenesis Kit was used to add a SacII restriction site downstream from the tetracycline resistance marker (primer SacII-downstream 5′ GTG CCA CCT GAC CCG CGG GTC TAA GAA ACC 3′ (SEQ ID NO:39)). The SpeI site and start codon were again removed from the multiple cloning site. QuikChange XL Site-Directed Mutagenesis Kit from Agilent Technologies was used to change the NheI cloning site in the leuCD cassettes to a SacII site (primers P503SacIIF 5′ GCG TAT CAC GAG GCC GCG GTT AAC GGA ACG C 3′ (SEQ ID NO:40) and P503SacIIR 5′ GCG TTC CGT TAA CCG CGG CCT CGT GAT ACG C 3′ (SEQ ID NO:41)). Vectors and leuCD cassettes were digested with SacII, ligated and transformed into E. coli using standard molecular biology techniques. Plasmids were screened by restriction digest and DNA sequencing. A representative vector map for P306leupMM1522+tet and a list of alternate vectors is shown in FIG. 29.

Sequencing of the modified vectors revealed the following nucleotide changes relative to the published sequences:

pMM1522: (See Example 12, infra. These differences were due to errors in the published sequence.):
Nucleotide positions 425-426-published sequence is AT, sequence from this study was TA.
Nucleotide position 5970-sequence from this study contained an additional adenine.
Nucleotide position 5981-sequence from this study was lacking a guanine present in the parent plasmid at 5981.
   pMM1522 minus tet:
Nucleotide position 5239 (non-coding region)-G5239A substitution.
   pHIS1522:
Nucleotide position 5916-sequence from this study contained an additional adenine.
Nucleotide position 5927-sequence from this study was lacking a guanine present in the parent plasmid at 5927.
   p3STOP1623 hp:
Nucleotide position 5124-sequence from this study contained an additional adenine.
Nucleotide position 5135-sequence from this study was lacking a guanine present in the parent plasmid at 5135.

### Example 12

3mCDTa2-pMM1522 Expression Plasmids

Screening of the constructs was initially performed using mutants of C. difficile binary toxin A (CDTa), which comprises specific point mutations designed to eliminate toxicity of the encoded protein. The mutant version of CDTa was 3mCDTa2, which comprises the following mutations: S345F, E385Q and E387Q (see Heinrichs et al., WO 2013/112867). 3mCDTa2 was codon optimized for expression in B. megaterium by Genscript (Piscataway, N.J.) and cloned into the B. megaterium expression vector pMM1522 using the BsrGI and SphI restriction sites (3mCDTa2-pMM1522). Expression of 3mCDTa2 was under the control of the PxylA. The expression levels of 3mCDTa2 in each of the leuCD constructs (Pleuleu (wt promoter), p306leu, p503leu and p201leu) was tested. Due to stability problems during synthesis of Pleuleu, an additional construct was created that had a single point mutation in the predicted −35 region of the promoter. This mutant promoter (called Pleu-1) was also tested. The constructs were also screened for their ability to grow in M9 minimal media.

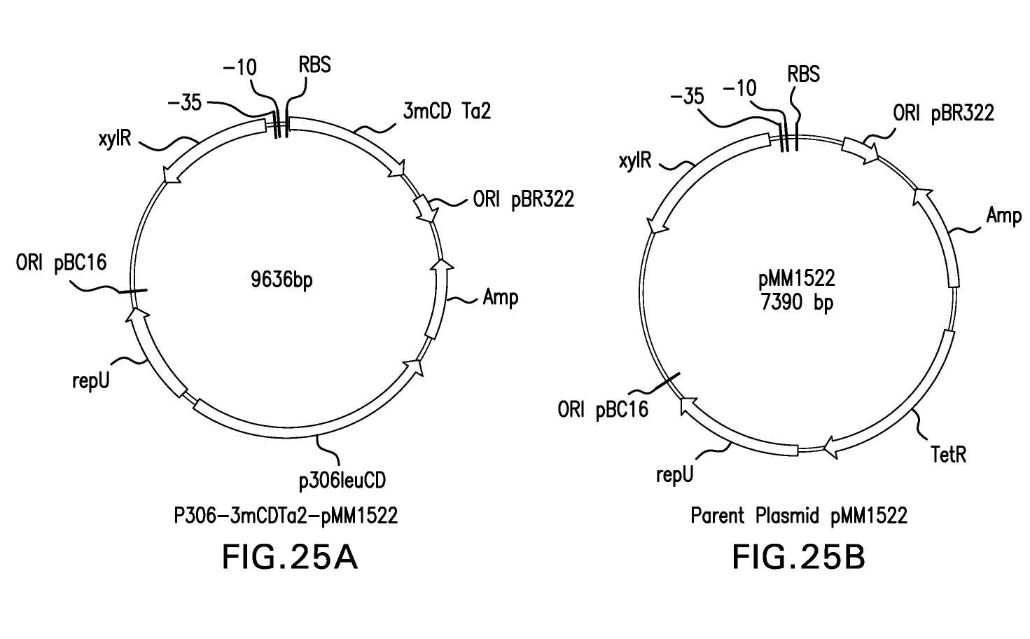

In order to create expression plasmids with leuCD and variant promoters, the tetracycline resistance marker in 3mCDTa2-pMM1522 was replaced with the leuCD constructs (see FIG. 25). Stratagene's QuikChange® Lightning Multi Site-Directed Mutagenesis Kit (Stratagene, Agilent Technologies, La Jolla, Calif.) was used to add SacII (primer SacII downstream 5′ GTG CCA CCT GAC CCG CGG GTC TAA GAA ACC 3′ (SEQ ID NO:42)) and NheI (primer NheI-upstream 5′ GAA CCC TGT TAC ATG CTA GCT CAT TAC ACT TC 3′ (SEQ ID NO:43)) restriction sites, which flanked the tetracycline resistance marker, and to remove a NheI site (primer pMM-NheI 5′ GCA TCG CCA GTC ACT ATG GCG TGC TGC AAG CGC TAT ATG CGT TGA TGC 3′ (SEQ ID NO:44)) present in the vector. After mutagenesis, vectors were screened by restriction digest with SacII and NheI. Once a vector was found that contained the correct mutations, the vector and the leuCD constructs were digested with SacII and NheI, and ligated and transformed into DH10B competent E. coli (Invitrogen) following standard techniques. Isolates were screened by restriction digest and plasmids with the correct restriction pattern were sent to Genewiz (South Plainfield, N.J.) for sequencing. There was a small gap in the sequence data upstream of the Pleuleu cassette for all plasmids.

Sequence analysis of the vectors indicated that there were a couple of nucleotide differences between the published sequence for the MoBiTec vectors and the pMM1522 vectors that were used for the expression plasmids. Specifically, nucleotide positions 425-426 of plasmid pMM1522 were reported to be AT in the published sequence, while sequence analysis revealed these nucleotide positions to be TA in all of the pMM1522 vectors used in these studies. Also, an additional adenine was present at nucleotide position 5970 and a guanine was lacking at position 5982 in the pMM1522 plasmids sequenced for this study relative to the published sequence. Our conclusion is that the sequence differences can be attributed to sequencing errors in the published sequence since later constructs contained the same nucleotide differences relative to the reported sequence.

### Example 13

Transformation of Δspo0AΔxylARΔleuCD D and Expression Studies

Protoplasts were made of Δspo0AΔxylARΔleuCD D following the procedure in Barg et al., 2005, supra. Plasmids P201-3mCDTa2-pMM1522, Pleu-1-3mCDTa2-pMM1522, Pleu-3mCDTa-pMM1522, and P306-3mCDTa2-pMM1522 were transformed into the protoplasts following the procedure in the MoBiTec's Bacillus megaterium Protein Production System Manual. Transformants were grown on M9 minimal media agar plates (Teknova, Hollister, Calif.). A very low transformation efficiency was observed. Only one colony grew on the P201leuCD-3mCDTa2-pMM1522 and four grew on the Pleu-1leuCD-3mCDTa2-pMM1522 plates. Five grew on the P306leuCD-3mCDTa2-pMM1522 plates and 4 grew on the Pleuleu-3mCDTa-pMM1522 plates.

Because of the poor growth observed in M9 minimal media, several different formulations of modified media were designed and tested. See Table 1. After formulating, all media were filter sterilized.

TABLE 1
Media for Growing Leucine Auxotroph Transformants

| Media Composition | OD600 PleuleuCD | OD600 P306leuCD |
|---|---|---|
| M9 media + glycerol + TE* | 3.302 | 3.032 |
| M9 salts + MgSO₄ + | 5.605 | 5.754 |
| CaCl₂ + glycerol + TE | | |
| M9 salts + MgSO₄ + | 0.4971 | 0.5821 |
| CaCl₂ + glycerol | | |
| M9 media + glycerol | 0.0759 | 0.0815 |
| M9 media + TE | 2.992 | 3.06 |
| M9 media | 0.0834 | 0.0963 |

*= Trace elements (TE's) solution 2. TE's were added post sterilization.

2×M9 salts were obtained from Invitrogen (Life Technologies, Carlsbad, Calif.). M9 media contains 2% glucose, 1 mM MgSO₄, and 0.1 mM CaCl₂. Supplements tested were as follows: 20 g/L glycerol, 1 mM MgSO₄, 0.1 mM CaCl₂, and 1.5 ml/L trace elements. Increased biomass was observed with media containing trace elements. An increase of biomass was also observed when glucose was replaced with glycerol. The best growth was observed using a medium containing 1×M9 salts, 1 mM MgSO₄, 0.1 mM CaCl₂, 20 g/L glycerol and trace elements, without glucose.

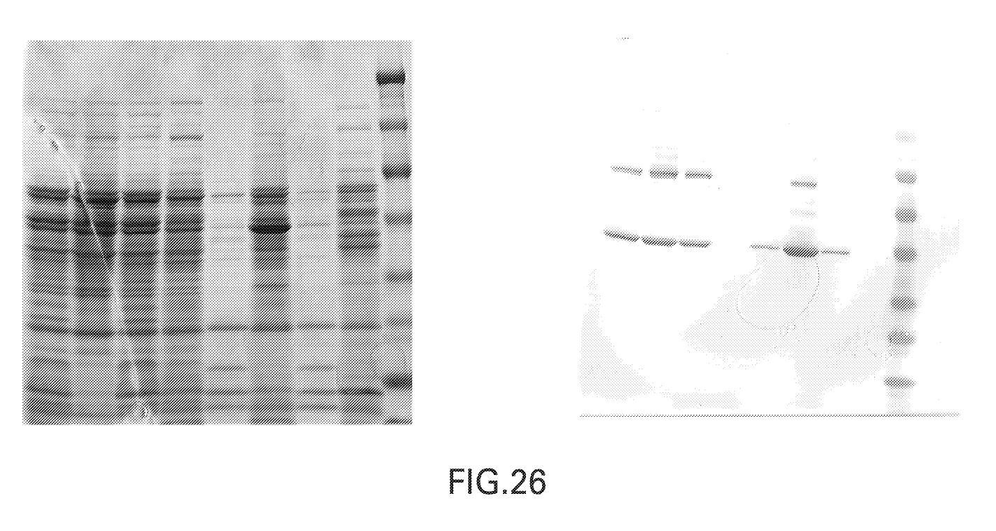

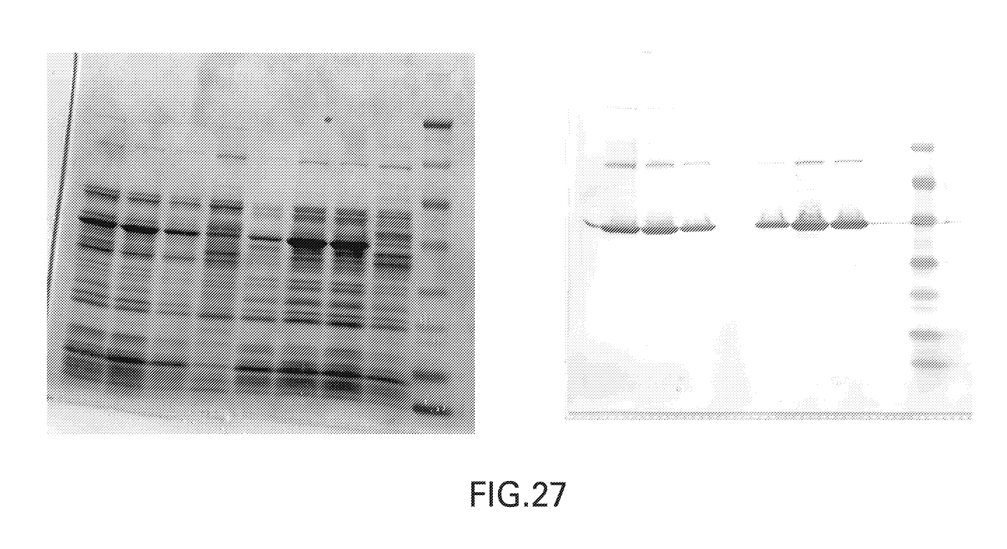

Expression studies were carried out to compare the expression levels of 3mCDTa2 in pMM1522 containing 4 different leuCD constructs. Expression plasmids tested were: P201leu-3mCDTa2-pMM1522, Pleu-1leu-3mCDTa2-pMM1522, Pleuleu-3mCDTa-pMM1522, and P306leu-3mCDTa2-pMM1522. Expression studies followed the protocol from MoBiTec except for the media. Two media were tested: modified M9 media (lx M9 salts, 1 mM MgSO₄, 0.1 mM CaCl₂, 20 g/L glycerol and trace elements) and LB plus 20 g/L glycerol. Expression was induced after 3 hours at an OD600 ˜0.5. P201leu-3mCDTa-pMM1522 grew poorly and was dropped from this study. Cultures were induced with 0.5% xylose for 5 hours and 20 hours at 37° C. Cells were harvested by centrifugation and cell pellets were stored overnight at −80° C. Expression was analyzed by Western blot and Coomassie stained gel (FIGS. 26 and 27). Similar expression levels were observed between the different constructs.

Qiagen QIAquick® Spin Minipreps (Qiagen N.V., Venlo, The Netherlands) were performed on the transformed Bacillus megaterium isolates. The plasmid from P201leu-3mCDTa2-pMM1522 was found to have undergone rearrangements (data not shown), so this construct was not included in further studies. The other plasmids Pleu-1leu-3mCDTa2-pMM1522, Pleuleu-3mCDTa-pMM1522, and P306leu-3mCDTa2-pMM1522 were verified by DNA sequencing (performed by Genewiz Inc., South Plainfield, N.J.).

1. An isolated or purified asporogenous Bacillus megaterium (B. megaterium) strain comprising a B. megaterium genome, wherein said genome is modified in that the spo0A gene is deleted or functionally deleted, wherein the strain does not produce spores.

2. The asporogenous strain of claim 1, wherein the spo0A gene is deleted from the B. megaterium genome.

3. The asporogenous strain of claim 1, wherein the B. megaterium genome is derived from a progenitor strain selected from DSM319, a DSM319 mutant strain, QMB1551, or a QMB1551 mutant strain.

4. The asporogenous strain of claim 1, wherein the B. megaterium genome is further modified by a deletion or functional deletion of the xylA and xylR genes, wherein the strain is unable to metabolize xylose.

5. The asporogenous strain of claim 4, wherein the xylA and the xylR genes are deleted from the B. megaterium genome.

6. The asporogenous strain of claim 1, wherein the B. megaterium genome further comprises a deletion or functional deletion of a gene that functions as part of the ilv-leu operon, wherein the strain is leucine auxotrophic.

7. The asporogenous strain of claim 6, wherein the B. megaterium genome comprises a deletion or functional deletion of the leuC and leuD genes.

8. The asporogenous strain of claim 7, wherein the leuC and the leuD genes are deleted.

9. The asporogenous strain of claim 1, further comprising at least one of an aacA-aphD resistance marker and a ermC resistance marker.

10. The asporogenous strain of claim 1, further comprising an expression vector, wherein the expression vector comprises a sequence of nucleotides that encodes a heterologous polypeptide, operatively liked to a promoter.

11. The asporogenous strain of claim 10, wherein the expression vector is pMM1522, pHIS1522, p3STOP1623 hp, or a derivative of pMM1522, pHIS1522, or p3STOP1623 hp.

12. The asporogenous strain of claim 10, wherein the expression vector comprises the leuC and leuD genes.

13. The asporogenous strain of claim 12, wherein the leuC and leuD genes comprise the nucleotide sequences set forth in SEQ ID NO:57 and SEQ ID NO:58.

14. The asporogenous strain of claim 12, wherein the leuC and leuD nucleotide sequences are operatively linked to an Ilv-leu promoter or variant thereof.

15. The asporogenous strain of claim 14, wherein leuC and leuD are operatively linked to an Ilv-leu variant promoter selected from the group consisting of: P503, P306, P201 and Pleu-1.

16. A promoter comprising a sequence of nucleotides as set forth in SEQ ID NO:7, SEQ ID NO:8, SEQ ID NO:9, or SEQ ID NO:59.

17. A method of producing a heterologous protein in a Bacillus megaterium (B. megaterium) host cell comprising:
(a) transforming a modified B. megaterium cell with an expression vector comprising a sequence of nucleotides that encodes the heterologous protein, wherein the modified cell comprises a B. megaterium genome that has been modified in that the spo0A gene is deleted or functionally deleted and wherein the modified cell does not produce spores;
(b) cultivating the transformed modified B. megaterium cell under conditions that permit expression of the nucleotide seqence to produce the heterologous protein; and
(c) optionally isolating the heterologous protein.

18. The method of claim 17, wherein the B. megaterium genome comprises a deletion or functional deletion of the xylA and xylR genes and cannot metabolize xylose.

19. The method of claim 17, wherein the B. megaterium genome further comprises a deletion or functional deletion of the leuC and leuD genes and the expression vector comprises a sequence of nucleotides that encodes leuC and a sequence of nucleotides that encodes leuD.

20. The method of claim 19, wherein leuC and leuD are operably linked to an ilv-leu promoter, or a variant ilv-leu promoter selected from the group consisting of: P503, P306, P201 and Pleu-1.
